(19) United States
(12) Patent Application Publication
(10) Pub. No.: US 2019/0224170 A1
(43) Pub. Date: Jul. 25, 2019

(54) THERAPIES AND METHODS TO TREAT INFLAMMATORY GASTROINTESTINAL DISEASES AND DISORDERS

(71) Applicant: The Regents of the University of California, Oakland, CA (US)

(72) Inventor: Hamid M. Said, Irvine, CA (US)

(21) Appl. No.: 16/255,723

(22) Filed: Jan. 23, 2019

Related U.S. Application Data
(60) Provisional application No. 62/621,551, filed on Jan. 24, 2018.

(51) Int. Cl. A61K 31/4188; A61K 31/197; A61P 1/00

(52) U.S. Cl. CPC A61K 31/4188; A61P 1/00; A61K 31/197

(57) ABSTRACT

The disclosure provides for therapies and methods to treat inflammatory gastrointestinal diseases and disorders with compositions that comprise therapeutically effective amounts of a biotin-based compound and/or a pantothenic acid-based compound.

### CROSS REFERENCE TO RELATED APPLICATIONS

This application claims priority under 35 U.S.C. § 119 from Provisional Application Ser. No. 62/621,551, filed Jan. 24, 2018, the disclosure of which is incorporated herein by reference.

### STATEMENT REGARDING FEDERALLY SPONSORED RESEARCH

This invention was made with Government support under Grant No. DK058057 awarded by the National Institutes of Health. The Government has certain rights in this invention.

### TECHNICAL FIELD

The disclosure provides for therapies and methods to treat inflammatory gastrointestinal diseases and disorders with compositions that comprise therapeutically effective amounts of a biotin-based compound and/or a pantothenic acid-based compound.

### BACKGROUND

The sodium-dependent multi-vitamin transporter (SMVT) is responsible for intestinal absorption of both vitamin B7 (biotin) and vitamin B5 (pantothenic acid). Both vitamins are essential for normal cellular functions and both must be obtained from exogenous sources as our body cannot synthesize these vitamins endogenously.

### SUMMARY

Intestinal absorption of the water-soluble vitamin biotin and pantothenic acid are carrier-mediated and involve the sodium-dependent multivitamin transporter (SMVT; product of the SLC5A6 gene). An SMVT-cKO mouse model was generated by an intestinal-specific (conditional) knockout of the mouse Slc5a6 gene (SMVT-cKO); and iSMVT-cKO knockout mouse was generated intestinal-specific (conditional) knockout of the mouse tamoxifen-inducible SLC5A6 gene. In the studies presented herein, both the SMT-cKO and iSMVT-cKO mice were associated with biotin deficiency, growth delay, and early death. Moreover, these mice had spontaneous and severe inflammation as well as abnormal histology in the large intestine together with altered gut permeability. Similar changes in gut integrity and immunity in normal mice were observed by feeding the mice a diet that was biotin deficient. It was further found in studies presented herein that biotin deficiency enhances the inflammatory responses in CD4⁺ T cells, which may contribute to inflammation associated with biotin deficiency.

It was further shown herein that biotin and pantothenic acid over-supplementation (BPS) could reverse the observed abnormalities in SMVT-cKO mice observed above. BPS was provided in the drinking water to mice before conception, dams during pregnancy and lactation, and to the SMVT-cKO mice throughout their life. The findings presented herein indicate that BPS use in SMVT-cKO mice prevented early death as well as normalization of the growth rate, intestinal integrity, pathology and inflammation. The findings presented herein provide clear evidence for a role for biotin and pantothenic acid in the maintenance of normal intestinal integrity and health. As such, use of biotin and pantothenic acid in combination can be used as therapy to treat inflammatory diseases of the gastrointestinal track, such as inflammatory bowel disease, coeliac disease, ulcerative colitis, Crohn's disease, enterocolitis, gastroenteritis, enteritis, and duodenitis.

Biotin (vitamin B7) acts as a cofactor for five carboxylases those are involved in a variety of metabolic reactions (e.g., fatty acid, glucose and amino acid metabolism; energy metabolism, reduction of cellular oxidative stress); it also plays a role in the regulation of gene expression. Emerging evidence has been also accumulating to show a important role for biotin in the normal functions of adaptive (T and B cells) as well as innate (DC) immune cells. The incidence of biotin deficiency and sub-optimal levels occurs in a variety of conditions (e.g., inflammatory bowel diseases, IBD and chronic alcoholism). On the other hand, supplementation with high doses of biotin has been associated with a distinctive positive clinical response in autoimmune/inflammatory disease, multiple sclerosis. Humans (mammals) obtain biotin from exogenous sources (diet and gut microbiota) via intestinal absorption. Absorption occurs via an efficient Na⁺-dependent, carrier-mediated process that involves the sodium-dependent multivitamin transporter, SMVT (product of the SLC5A6 gene). SMVT also transports pantothenic acid and lipoate. In a particular embodiment, the disclosure provides a method of treating a subject with an inflammatory gastrointestinal disease or disorder comprising administering a therapeutically effective amount of a composition comprising, consisting essentially of, or consisting of a biotin-based compound and a pantothenic acid-based compound. In a further embodiment, the biotin-based compound comprises a structure of Formula I(a):

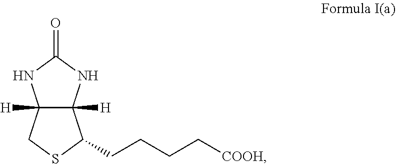 [SMILES: O=C(O)CCCC[C@@H]1SC[C@@H]2NC(=O)N[C@@H]21]

or a pharmaceutically salt, prodrug, or solvate thereof. In yet a further embodiment, the composition comprises 1 mM or equivalent thereof of the biotin-based compound. In another embodiment, the pantothenic acid-based compound comprises a structure of Formula II:

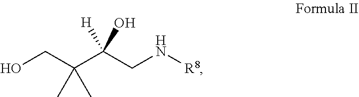

or a pharmaceutically salt, prodrug, or solvate thereof, wherein, R⁸ is selected from the group consisting of

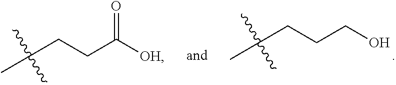 [SMILES: *CCC(=O)O ; *CCCO]

In a further embodiment, the pantothenic acid-based compound comprises a structure of Formula II(a):

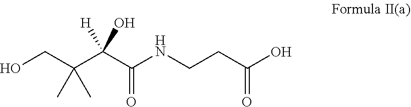

or a pharmaceutically salt, prodrug, or solvate thereof. In yet a further embodiment, the composition comprises at least 1 mM or equivalent thereof of the pantothenic acid-based compound. In a certain embodiment, the subject is a human subject. In another embodiment, the inflammatory gastrointestinal disease or disorder is selected from the group consisting of inflammatory bowel disease, coeliac disease, ulcerative colitis, Crohn's disease, enterocolitis, gastroenteritis, enteritis, and duodenitis. In a particular embodiment, the inflammatory gastrointestinal disease or disorder is inflammatory bowel disease. In a further embodiment, the composition consists essentially of biotin and pantothenic acid. In yet a further embodiment, the composition consists of biotin and pantothenic acid. In another embodiment, the composition is formulated for oral administration. In yet another embodiment, the composition is formulated as an extended release oral formulation. In a further embodiment, the composition is formulated as a tablet, a capsule, a liquid filled capsule, or a gelatin-based chewable composition. In a particular embodiment, the composition is administered in combination with one or more drugs or therapeutic agents used to treat an inflammatory gastrointestinal disease or disorder. Examples of drugs or therapeutic agents used to treat inflammatory gastrointestinal diseases or disorders include, but are not limited to, anti-inflammatory drugs, immunosuppressant drugs, antibiotics, tumor necrosis factor (TNF)-alpha inhibitors, biologics and pain relievers. Examples of anti-inflammatory drugs include but are not limited to, corticosteroids and aminosalicylates. Examples of corticosteroids include, but are not limited to, prednisolone, prednisone, hydrocortisone, methylprednisolone, beclometasone dipropionate, and budesonide. Examples of immunosuppressant drugs include, but are not limited to, azathioprine, mercaptopurine, cyclosporine, and methotrexate. Examples of biologics include, but are not limited to, infliximab, adalimumab, golimumab, natalizumab, vedolizumab, and ustekinumab. In a further embodiment, administration of the composition provides for one of more affects selected: (1) decreased gastrointestinal inflammation; (2) improvement in patient-reported outcomes as to the status of the patient's health condition; (3) improvement in the endoscopic appearance of mucosa; and/or (4) decreased appearance of blood in a stool.

In a certain embodiment, the disclosure also provides a method to decrease gastrointestinal inflammation caused by CD4′T cells in a subject in need thereof, comprising administering to the subject a composition consisting essentially of a biotin-based compound and a pantothenic acid-based compound. In a further embodiment, administration of the composition results in decreased expression of mTOR and/or p70S6. In yet another embodiment, administration of the composition results in the decreased expression of inflammatory cytokines. Examples of inflammatory cytokines include but are not limited to IFN-γ, TNF, and IL-17.

### DESCRIPTION OF DRAWINGS

FIG. 1A-D demonstrates that biotin deficiency enhances proinflammatory cytokine secretion in anti-CD3/CD28-stimulated CD4⁺ T cells. CD4⁺ T cells were stimulated with CD3/CD28 magnetic beads and cultured in biotin-deficient and -sufficient AIM V medium for 24 and 72 h. (A) Bar graph depicts the levels of IFN-γ, TNF, IL-17, and IL-10 in the supernatants of CD4⁺ T cells cultured in biotin+ve (sufficient) and biotin-ve (deficient) media. (B) Bar graphs depict the levels of TFs T-bet, RORγt, Foxp3, and GATA-3 in the cells, as determined by quantitative real-time PCR. GAPDH was used as control. (C) Dot plots depict the percentages of CD4⁺CD25⁺Foxp3⁺ Tregs in unstimulated cells and cells stimulated for 72 h. (D) Bar graph depicts the cumulative percentage of Tregs. Data are mean±SE of six experiments with different donors. *p≤0.05, **p≤0.01.

FIG. 2A-C shows that biotin deficiency enhances the differentiation of naive and memory CD4⁺ T cells toward Th1 and Th17 cells. Naive and memory CD4⁺ T cells were supplemented with factors inducing Th1, Th17, and Treg differentiation for 3 d (memory) and 6 d (naive). Intracellular staining for cytokines was performed after PMA and ionomycin stimulation. Culture supernatants was harvested, and the level of IFN-γ, IL-10, and IL-17 cytokines was evaluated by ELISA. (A) Th1 differentiation. (B) Th17 differentiation. (C) Treg differentiation. Data are mean±SE of five experiments with different donors. *p<0.05, **p<0.01.

FIG. 3A-B demonstrates that biotin deficiency decreases the proliferation CD4⁺ T cells. CFSE-labeled CD4⁺ T cells were cultured in biotin-deficient and control medium for 72 h. (A) Dot plots depict the dilution of CFSE dye as a measure of proliferation. (B) CD4⁺ T cells cultured under biotin-deficient and -sufficient conditions for 72 h were stained with 7-AAD to determine cell death. Dot plots depict the percentage of 7-AAD⁺ T cells in the culture. Bar graph depicts the same. Data are mean±SE of four experiments. *p<0.05; **p<0.01.

FIG. 4A-C demonstrates that Biotin deficiency enhances the expression of phospho-mTOR, p70S6 kinase, and proinflammatory cytokines in anti-CD3/CD28-stimulated CD4⁺ T cells. CD4⁺ T cells were stimulated with anti-CD3/CD28 magnetic beads and cultured in biotin-deficient and -sufficient AIM V medium, with or without rapamycin (100 ng/ml), for 3 d (72 h). Intracellular levels of phospho-mTOR and p70S6 kinase were determined by flow cytometry. (A) Phospho-mTOR expression. (B) Phospho-p70S6 kinase expression. (C) Proinflammatory cytokines in control (B+ve), biotin-deficient (B-ve), B+ve with rapamycin, and B-ve with rapamycin CD4⁺ T cells. Data are mean±SE of five experiments with different donors. *p<0.05; **p<0.01.

FIG. 5A-E shows that biotin deficiency enhances inflammation in the cecum and alters the expression of Th TFs and phospho-mTOR in inguinal lymph nodes of mice. C57BL/6J wild-type mice were fed a biotin-sufficient or -deficient diet for 16 wk. Sections of the cecal wall were embedded in paraffin and stained with H&E (original magnification ×40). (A) Infiltration of neutrophils and CD4⁺ and CD8⁺ T cells in biotin-deficient and pair-fed control mice (DAB stain). (B) Number of CD4⁺ and CD8⁺ T cells in control and biotin-deficient mice cecum. (C) Inguinal lymph nodes were harvested from diet-induced biotin-sufficient and -deficient mice, and single-cell suspensions were prepared. Expression of TFs T-bet, RORγt, Foxp3, and GATA-3 was evaluated by quantitative real-time PCR. (D) Expression of phospho-mTOR. (E) Proinflammatory cytokine level in inguinal lymph nodes isolated from diet-induced biotin-deficient and pair-fed control mice. Data are mean±SE of five mice. *p≤0.05, **p≤0.01.

FIG. 6A-D demonstrates the effects of biotin and pantothenic acid over-supplementation (BPS) on SMVT-cKO mice growth rate, body weight, bone density and biotin status. (A) (left panel, i) Representative image of SMVT-cKO mouse (left) and its sex-matched BPS WT-littermate (right) showing clear phenotypic differences in overall size and length. (Right panel, ii) Representative image of BPS SMVT-cKO mouse (left) and its sex-matched BPS WT littermate (right) showing no difference in size and length. (B) (left panel, i) Representative X-ray image of a SMVT-cKO mouse (left) and its sex-matched WT littermate (right) showing distinct difference in bone length. (right panel, ii) Representative X-ray image of BPS SMVT-cKO mice (left) and its BPS WT littermate (right) showing no difference in bone size and length. (C) Growth chart showing no difference in weight gain of BPS SMVT-cKO mice compared with their BPS WT littermate. (D) Level of total biotinylated proteins in the liver (a measure of biotin status) of BPS SMVT-cKO mice and their BPS WT littermates. Data are means±SE of at least three separate sets of mice. Abbreviations: BPS, biotin and pantothenic acid supplementation; cKO, conditional knockout of the mouse Slc5a6 gene; NS, not significant; SMVT, sodium-dependent multivitamin transporter; WT, wild type.

FIG. 7A-F provides the results of various experiments with BPS in SMVT-cKO mice and wild type mice. (A) Representative section of the cecum of BPS WT littermate (left, i) and BPS SMVT-cKO mouse (right, ii) (hematoxylin and eosin, ×40). Animals of both groups showed normal cecal morphology. (B) Effect of BPS to SMVT-cKO mice on intestinal permeability. Intestinal permeability was determined using 4-kDa FITC-dextran method. Data are means±SE of three pairs of BPS SMVT-cKO and WT littermates. (C) Effect of BPS to SMVT-cKO mice on the level of mRNA expression of TJ proteins and MLCK in the cecum. mRNA levels were determined by real-time RT-PCR, and data were normalized relative to β-actin. Data are means±SE of at least three pairs of BPS SMVT-cKO mice and WT littermates. (D) Effect of BPS to SMVT-cKO mice on the level of protein expression of TJ proteins and MLCK in the cecum. Protein levels were determined by Western blot analysis, and data were normalized relative to β-actin expression as described in Methods. The graphs show relative protein expression of claudin-1 (i), claudin-2 (ii), ZO-1 (iii), and MLCK (iv) in cecum samples of SMVT-cKO mice and their WT littermates. Data are means±SE of at least three separate sets of mice. (E) Expression of mRNA of mucin genes in the cecum of SMVT-cKO mice and their WT littermates. mRNA levels were determined by real-time RT-PCR, and data were normalized relative to villin. Data are means±SE of at least three separate sets of mice (*P<0.05; **P<0.01; NS, not significant). (F) Effect of BPS to SMVT-cKO mice and their WT littermates on mRNA expression of mucins in the cecum. Level of mRNA expression of MUC-1 (i), MUC-2 (ii), and MUC-3 (iii) in cecum of BPS SMVT-cKO mice and their sex-matched BPS WT littermates. Data are means±SE of at least three sets of mice. Abbreviations: BPS, biotin and pantothenic acid supplementation; cKO, conditional knockout of the mouse Slc5a6 gene; MLCK, myosin light chain kinase; MUC, mucin; SMVT, sodium-dependent multivitamin transporter; TJ, tight junction; WT, wild type; ZO-1, zonula occludens 1.

FIG. 8A-C provides the results of various experiments with BPS in SMVT-cKO mice and wild type mice on the expression of cytokines. (A) Effect of BPS of SMVT-cKO mice on the level of mRNA expression of proinflammatory cytokines in the cecum. mRNA levels were determined by real-time RT-PCR, and data were normalized relative to β-actin. Data are means±SE of at least three separate sets of mice (NS, not significant). (B) Expression of mRNA of oxidative stress-responsive genes in the cecum of SMVT-cKO mice and their WT littermates. mRNA levels were determined by real-time RT-PCR and data were normalized relative to β-actin. Data are means±SE of at least three separate sets of mice (**P<0.01; NS, not significant). (C) Effect of BPS to SMVT-cKO mice and their WT littermates on mRNA expression of oxidative stress-responsive genes in the cecum. mRNA levels were determined by real-time RT-PCR, and data were normalized relative to β-actin. Data are means±SE of at least three separate sets of mice. Abbreviations: BPS, biotin and pantothenic acid supplementation; cKO, conditional knockout of the mouse Slc5a6 gene; FMO, flavin-containing monooxygenase; LPO, lipid peroxidation; NOS, nitric oxide synthase; SMVT, sodium-dependent multivitamin transporter; SOD, superoxide dismutase; WT, wild type.

FIG. 9 presents a schematic of the process used to develop a tamoxifen-inducible SMVT conditional (intestine-specific) knockout (iSMVT-cKO) in adult mice, which was used for further characterization of the role of biotin in normal intestinal homeostasis and immunity.

### DETAILED DESCRIPTION

As used herein and in the appended claims, the singular forms “a,” “an,” and “the” include plural referents unless the context clearly dictates otherwise. Thus, for example, reference to a “B-vitamin” includes a plurality of B-vitamins and reference to “an inflammatory gastrointestinal disease or disorder” includes reference to one or more inflammatory gastrointestinal diseases or disorders and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. Moreover, with respect to any term that is presented in one or more publications that is similar to, or identical with, a term that has been expressly defined in this disclosure, the definition of the term as expressly provided in this disclosure will control in all respects.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

A “disorder” or “disease” is any condition that would benefit from treatment with the compositions and/or methods of the disclosure. An example of disorders and diseases that can be treated with the compositions and/or methods disclosed herein, includes inflammatory gastrointestinal diseases and disorders.

An “effective amount” refers to an amount effective, at dosages and for periods of time necessary, to achieve the desired therapeutic or prophylactic result.

An “individual,” “subject,” or “patient” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal refers to a human patient. In particular, a human patient in need of treatment of an inflammatory gastrointestinal disease and disorder.

The term “substantially similar” or “substantially the same,” as used herein, denotes a sufficiently high degree of similarity between two numeric values (for example, one associated with an antibody of the disclosure and the other associated with a reference/comparator antibody), such that one of skill in the art would consider the difference between the two values to be of little or no biological and/or statistical significance within the context of the biological characteristic measured by the values (e.g., K_{d} values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{d} values). The difference between said two values is, for example, greater than about 10%, greater than about 20%, greater than about 30%, greater than about 40%, and/or greater than about 50% as a function of the value for the reference/comparator molecule.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease; alleviation of symptoms; diminishment of any direct or indirect pathological consequences of the disease; decreasing the rate of disease progression; amelioration or palliation of the disease state; and remission or improved prognosis. In some embodiments, a biotin-based compound and a pantothenic acid-based compound of the disclosure are used to delay development of a disease or disorder, e.g., an inflammatory gastrointestinal diseases and disorder.

The sodium-dependent multivitamin transporter (SMVT) is a product of the SLC5A6 gene and is responsible for the transport of the water-soluble vitamins biotin and pantothenic acid, as well as the metabolite lipoate. Biotin is an essential micronutrient that cannot be synthesized endogenously by mammalian cells and plays an important role in normal cellular metabolism, growth, and development. The vitamin is a coenzyme for multiple carboxylases involved in a variety of metabolic pathways, such as fatty acid synthesis, gluconeogenesis, and catabolism of branched chains amino acids. Biotin also plays a role in energy metabolism (i.e., ATP production), cellular oxidative stress regulation and the expression of many genes. In addition, a role for biotin in normal immune function has been well documented. More recent findings have shown that biotin also plays a role in influencing the colonization/invasiveness of certain enteropathogenic bacteria, and in mediating the effect of probiotic bacteria on gut microbial community. Deficiency/suboptimal levels of biotin occur in a variety of conditions and lead to serious clinical abnormalities which include neurological and dermatological disorders, as well as growth retardation. With regard to pantothenic acid, this vitamin also cannot be synthesized endogenously by mammalian cells. This micronutrient is required for the biosynthesis of coenzyme A and acyl carrier protein in mammalian cells, and therefore, it is important for carbohydrate, fat, and to a lesser extent protein metabolism. Spontaneous pantothenic acid deficiency has not been reported in humans. With regard to lipoate, this substrate acts as an antioxidant, and mammalian cells can synthesize a considerable amount of this substrate endogenously.

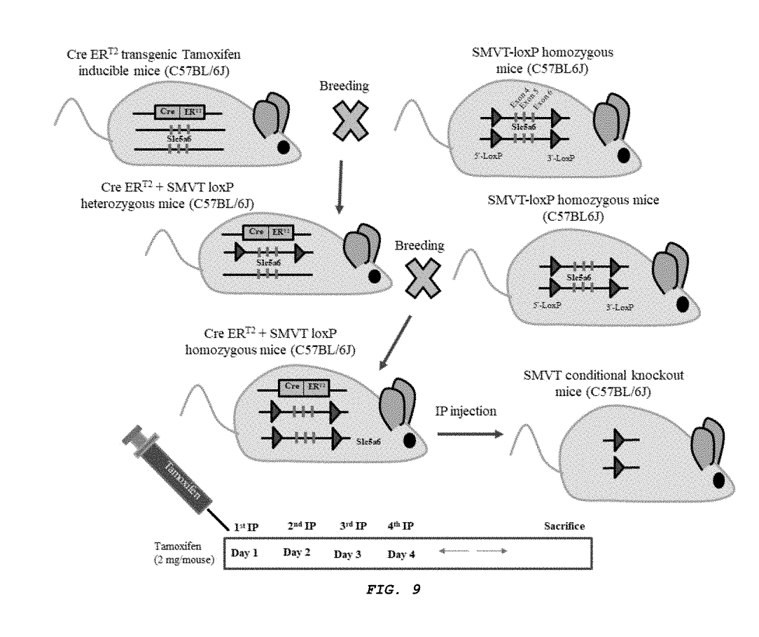

A conditional (intestinal-specific) SMVT knockout (SMVT-cKO) mouse model (using the Cre-Lox approach); and a conditional intestinal-specific (conditional) knockout of the mouse tamoxifen-inducible SLC5A6 gene (also using the Cre-Lox approach (see FIG. 9)) were generated and used to determine the relative contribution of the SMVT system toward intestinal carrier-mediated biotin uptake. The findings indicated that the SMVT is the only biotin transport system that operates in the intestine, and that its knockout leads to biotin deficiency. It was also observed that all of the SMVT-cKO exhibited growth retardation and died within the first 10 weeks of life; and the iSMVT-cKO mice develop biotin deficiency and a spontaneous chronic intestinal inflammation in the small and large intestine of adult mice similar to SMVT-cKO mice. Furthermore, the knockout animals developed severe chronic active inflammation with abnormal histology in the large intestine, reminiscent of that seen in ulcerative colitis in humans, including focal cryptitis/crypt abscesses, focal low-grade adenomatous changes, and extensive submucosal edema. Further investigations of the observed intestinal inflammation in the SMVT-cKO mice showed (1) an increase in the level of expression of proinflammatory cytokines (i.e., TNF-α, and IFN-γ), (2) an increase in gut permeability, and (3) changes in the level of expression of important tight junction (TJ) proteins compared with their wild-type (WT) littermates. Investigations in the iSMVT-cKO mice exhibited comparable results, including (1) an increase in the level of expression of proinflammatory cytokines (i.e., TNF-α, IL-1β, and IL-6), (2) induction in the level of expression of calprotectin genes and mucin genes; (3) reduction in the expression of Lgr5; (4) an increase in gut permeability, and (5) changes in the level of expression of important tight junction (TJ) proteins compared with their wild-type (WT) littermates. Similar changes in gut integrity and immunity were observed in mice made biotin deficient via dietary means. The latter suggests an important role for biotin in the development of the intestinal abnormalities observed in the SMVT-cKO mice and iSMVT-cKO mice, and pointed to a possible role for the vitamin in the maintenance of normal gut physiology.

In further studies presented herein looking at the mechanism of biotin deficiency in intestinal inflammation, it was found that under biotin-deficient conditions, innate immune system cells (dendritic cells) produce increased levels of proinflammatory cytokines. The dendritic cells cultured under biotin-deficient conditions also primed T cells toward inflammatory Th1/Th17 responses. CD4⁺ T lymphocytes played a key role in the induction and regulation of inflammatory responses. They can be divided into four major subsets (Th1/Th2/Th17/T regulatory cells [Tregs]) based on the nature of their cytokine secretion and expression of signature transcription factors (TFs). Th1 cells primarily secrete IFN-γ under the control of the TF T-bet, whereas Th17 cells express the TF RORγt, and IL-17 is the major cytokine secreted by these cells. Th1 and Th17 subsets are considered highly inflammatory and have been demonstrated to have a major role in inducing autoimmune and inflammatory disorders, such as inflammatory bowel disease, multiple sclerosis, and rheumatoid arthritis. In contrast, Th2 cells are positive for the TF GATA-3 and produce cytokines IL-4 and IL-5, which are key players in allergic responses. Tregs express TF Foxp3 and act as regulators of inflammation. The major function of Tregs is to suppress inflammation via secretion of anti-inflammatory cytokines IL-10 and TGF-β. Enhancing the induction of Tregs in autoimmune, inflammatory, and allergic disorders is considered an effective mode of treatment. In view of CD4⁺ T lymphocytes important role in inflammatory responses, studies were conducted herein on the effect of biotin deficiency on human CD4⁺ T lymphocyte function in vitro and in vivo in mice. In particular, the studies presented herein revealed that anti-CD3/CD28-stimulated CD4⁺ T cells cultured in biotin-deficient medium secreted significantly enhanced levels of the proinflammatory cytokines IFN-γ, TNF, and IL-17. Expression of the transcription factors T-bet and RORγt was increased, whereas Foxp3 expression was decreased, in biotin-deficient CD4⁺ T cells. The percentage of T regulatory cells was also decreased under biotin-deficient condition. A similar increase in T-bet, RORγt, and proinflammatory cytokine levels, as well as a decrease in Foxp3, was observed in inguinal lymph nodes of mice fed a biotin-deficient diet relative to pair-fed controls. Furthermore, differentiation of CD4⁺ T cells toward Th1 and Th17 cells was also enhanced. In vitro and in vivo investigations indicated that the increased inflammatory response was due to enhanced activation of the mammalian target of rapamycin signaling pathway in biotin-deficient CD4⁺ T cells. Accordingly, the studies presented herein indicate that biotin deficiency enhances the inflammatory responses in CD4⁺ T cells, which may contribute to inflammation associated with biotin deficiency.

Further, studies were performed herein where high doses of biotin and pantothenic acid (BPS) were orally administered, via drinking water, to dams during pregnancy and lactation and to the SMVT-cKO mice throughout their life in order to determine if such over-supplementation could reverse the above-described intestinal abnormalities using animal models. BPS was initiated before conception and the regimen was continued throughout pregnancy and lactation of the dams; and throughout the life of the offspring to ensure that sufficient biotin and pantothenic acid were provided to the SMVT-cKO animals at all times. The results showed that such supplementation normalizes all of the above-described abnormalities, i.e., early death of the animals, impairment in their growth rate and skeletal development as well as the development of inflammation and abnormal pathology in their intestine. The abnormalities in gut permeability, level of expression of important TJ proteins and MLCK, the mucosal inflammation, and the changes in the level of expression of proinflammatory cytokines and stress response genes in the SMVT-cKO animals were all normalized by the administration of BPS. These findings clearly demonstrate the important role that biotin and pantothenic acid plays in maintaining normal intestinal integrity and health. In mice made biotin deficient via dietary means, similar changes in large intestinal integrity, combined with the development of spontaneous inflammation were also observed. Of relevance to the findings in the current disclosure is the recent identification of loss of function mutations in the SLC5A6 gene in a 15-month old child who demonstrated growth impairment, variable immunodeficiency, and bone abnormalities, among other symptoms. It was found that the child's condition significantly improved following administration of high doses of biotin and pantothenic acid. Accordingly, the findings presented herein provide evidence for the important role that biotin and pantothenic acid play in the maintenance of normal intestinal integrity and physiology.

Further studies presented herein indicate that administration of therapeutically effective doses of biotin and pantothenic acid obviated abnormalities seen with SMVT-cKO animals (e.g., impairment in growth rate and skeletal development as well as the development of intestinal inflammation and pathology) and early death of the SMVT-cKO animals. In particular, abnormalities that were observed in gut permeability; in the level of expression of important tight junction (TJ) proteins and Myosin light chain kinase (MLCK); in mucosal inflammation; in the level of expression of pro-inflammatory cytokines; and in the level of expression of stress response genes in the SMVT-cKO animals were all normalized by the administration of therapeutically effective doses of biotin and pantothenic acid. Thus, the findings presented herein, clearly establishes that therapeutically effective doses of biotin and pantothenic acid can be used to treat subjects with an inflammatory gastrointestinal disease or disorder, e.g., inflammatory bowel disease, coeliac disease, ulcerative colitis, Crohn's disease, enterocolitis, gastroenteritis, enteritis, and duodenitis.

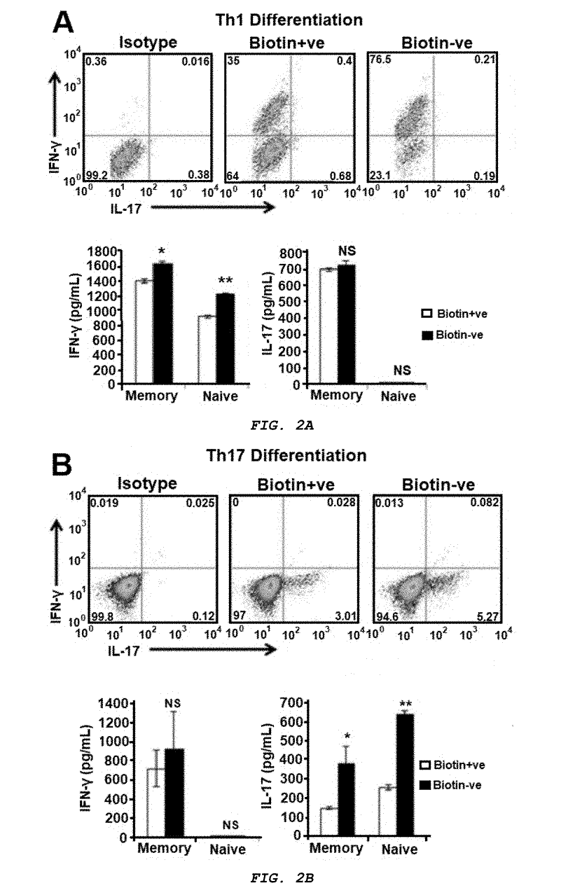

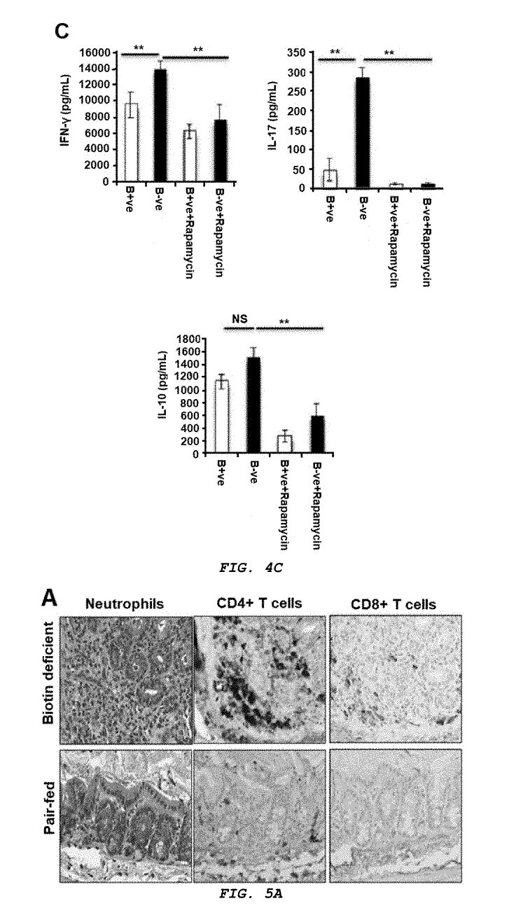

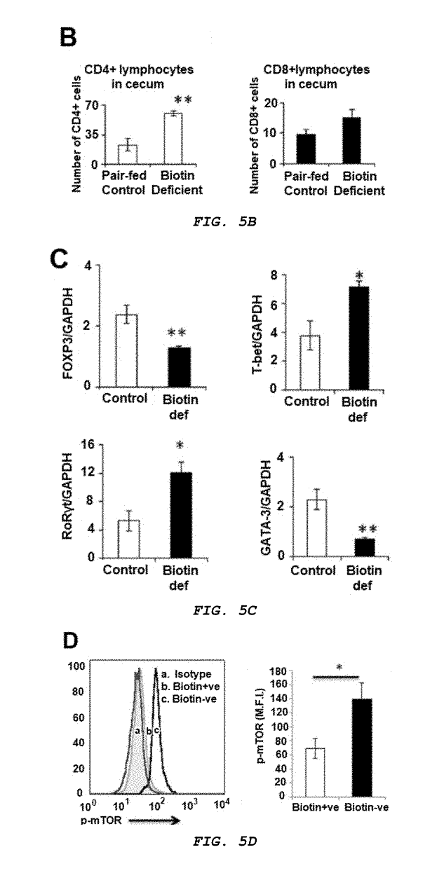

Additional investigations presented herein revealed that human CD4⁺ T cells cultured under biotin-deficient conditions develop an inflammatory phenotype. These cells display enhanced secretion of IFN-γ, TNF, and IL-17 proinflammatory cytokines, as well as an increase in the expression of the TFs T-bet and RORγt. The differentiation of naive and memory T cells toward Th1 and Th17 conditions was also enhanced (see FIG. 2A, 2B). Concomitantly, biotin deficiency decreased the expression of Foxp3 cells and the number of Tregs. Thus, it appears that biotin deficiency shifts the balance of Th cell responses toward Th1 and Th17 inflammatory cells and away from Tregs. Th1 and Th17 cells are well established as significant players in Crohn's disease and colitis. The accumulation of CD4⁺ T cells in the intestine of these mice compared with normal mice confirms the role of biotin deficiency in immune-mediated intestinal inflammation (see FIG. 5A). In addition, increased expression of T-bet and RORγt and decreased Foxp3 expression in inguinal lymph nodes of diet-induced biotin-deficient mice suggest that the effect of biotin deficiency is not localized to the intestine, which is the site of biotin absorption, but, rather, is systemic. Therefore, biotin deficiency may be involved in the induction of autoimmune and inflammatory disorders throughout the body. Th1 and Th17 cells also play a major role in the pathology of other autoimmune disorders, such as rheumatoid arthritis. Recent articles have suggested an association between biotin deficiency and multiple sclerosis, an autoimmune disorder of the nervous system. Thus, increased Th1 and Th17 cell numbers under biotin deficiency may be one of the mechanisms involved in the increased inflammation associated with biotin deficiency. Recent studies pointed out that, in the absence of mTOR signaling, mouse CD4⁺ T cells were unable to differentiate into Th1 and Th17 cells in vitro or in vivo; however, the tendency of CD4⁺ T cells to differentiate toward the Th2 subset remains unaltered. mTOR may also be required for differentiation of human T cells toward Th17 cells, highlighting the importance of this pathway in the differentiation of T cells toward the inflammatory phenotype. mTOR-deficient murine naive CD4⁺ T cells failed to differentiate into any Th lineage but rather differentiated toward inducible Tregs. Dysfunction of mTOR signaling has been reported in various human autoimmune diseases. mTOR was also found to be activated in peripheral T cells in patients with systemic lupus erythematosus. Rapamycin is an inhibitor of mTOR signaling; an in vitro study revealed inhibition of IL-17 expression and increased expression of Tregs in systemic lupus erythematosus patients after exposure to rapamycin. Collectively, these observations point out the important role of mTOR in the induction of inflammatory CD4⁺ T cells. Therefore, increased mTOR and p70S6 kinase expression, as was observed in the current investigation in CD4 T cells cultured under biotin-deficient conditions in vitro and in vivo (see FIGS. 4 and 5, respectively), might be responsible for the enhanced secretion of inflammatory cytokines IFN-γ, TNF, and IL-17 and the decreased percentages of Foxp3-expressing Tregs.

Accordingly, the findings presented herein show that biotin deficiency enhances the proinflammatory response in human CD4⁺ T lymphocytes. It also leads to the activation of mTOR signaling in CD4⁺ T cells, which leads to the production of increased levels of proinflammatory cytokines IFN-γ, TNF, and IL-17 and decreased expression of Tregs. Thus, the data presented herein provide for a possible mechanism for the active inflammation in the gut induced by biotin deficiency.

In a particular embodiment, the disclosure provides for treating a subject with an inflammatory gastrointestinal disease or disorder with a composition comprising a biotin-based compound and a pantothenic acid-based compound at therapeutically effective amounts. In a further embodiment, the composition of the disclosure comprises, consists essentially of, or consists of a biotin-based compound that has the structure of Formula I:

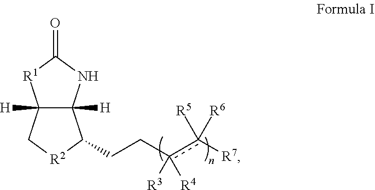

or a pharmaceutically acceptable salt, prodrug, or solvate thereof, wherein,

R¹ is NH or CH₂;

R² is S or O;

R³-R⁶ are independently selected from H, D, halo, (C₁-C₃) alkyl, OH, NH₂, and SH;

R⁷ is selected from OH, NH₂, —COH, —COOH, —COOCH₃, —COOCH₂CH₃, and —COCH₃; and

n is 0, 1, 2, 3, 4, or any range that includes or is between any two of the foregoing values.

In a further embodiment, the disclosure provides for a composition which comprises, consists essentially of, or consists a biotin-based compound having the structure of Formula I(a):

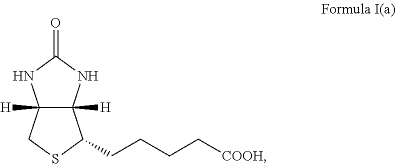 [SMILES: O=C(O)CCCC[C@@H]1SC[C@@H]2NC(=O)N[C@@H]21]

or a pharmaceutically acceptable salt, prodrug, or solvate thereof.

In a certain embodiment, the composition of the disclosure comprises a pantothenic acid-based compound that has the structure of Formula II:

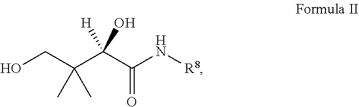

or a pharmaceutically acceptable salt, prodrug, or solvate thereof, wherein R⁸ is selected from

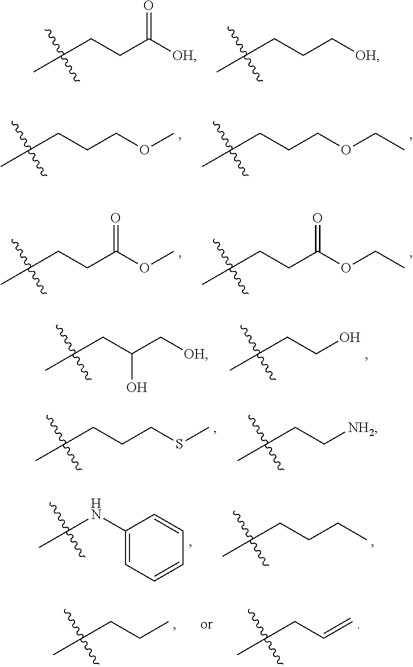

In a further embodiment, the composition of the disclosure comprises a pantothenic acid-based compound that has the structure of Formula II(a):

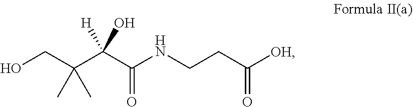

or a pharmaceutically acceptable salt, prodrug, or solvate thereof.

While it may be possible for the compounds of the disclosure to be administered as raw chemicals, it is also possible to present them as a pharmaceutical composition. Accordingly, provided herein are pharmaceutical compositions which comprise a biotin-based compound and a pantothenic acid-based compound disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The compositions of disclosure include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a biotin-based compound and a pantothenic acid-based compound of the disclosure or pharmaceutically salts, prodrugs, or solvates thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

The compositions include those suitable for oral administration. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; as an oil-in-water liquid emulsion or a water-in-oil liquid emulsion; or as a chewable formulation (e.g., a gelatin based gummy). The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

In certain embodiments, surfactants are selected from the group consisting of Tween 80, sodium lauryl sulfate, and docusate sodium.

In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

In certain embodiments, sustained release polymers are selected from the group consisting of POLYOX® (poly(ethylene oxide), POLYOX® N60K grade, Kollidon® SR, HPMC, HPMC (high viscosity), HPC, HPC (high viscosity), and Carbopol®.

In certain embodiments, extended/controlled release coating are selected from a group of ethylcellulose polymers, such as ETHOCEL™ and Surelease® Aqueous Ethylcellulose Dispersions.

In certain embodiments, antioxidants are selected from a group consisting of butylated hydroxyanisole (BHA), butylated hydroxytoluene (BHT), sodium ascorbate, and α-tocopherol.

In certain embodiments, tablet coatings are selected from the group of Opadry® 200, Opadry® II, Opadry® fx, Opadry® amb, Opaglos® 2, Opadry® tm, Opadry®, Opadry® NS, Opalux®, Opatint®, Opaspray®, Nutraficient®.

Preferred unit dosage formulations are those containing an effective dose, as herein below recited, or an appropriate fraction thereof, of the active ingredient.

The biotin-based compound and the pantothenic acid-based compound may each be administered orally at a dose per day of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg or any range that includes or is between any two of the foregoing values, including fractional increments thereof. The dose range for adult humans is generally from 10 mg to 100 mg/day. For example, children with genetic defects in biotin metabolism or biotin dependent enzymes, the dose of biotin can be between 10 and about 60 mg/day. In another embodiment, assuming body size of 40 Kg, a desired dose can be administered to achieve a serum concentration of about of 10-60 micromol/L. This serum concentration is greater than a 1000 fold higher than the normal serum level of 0.133-0.329 nmol/L. At this higher concentration, biotin will generally enter cells via simple diffusion. Biotin is non-toxic and excess biotin is excreted in the urine. Thus, one or more (e.g., 1-6) tablets of 10 mg biotin can be used. Tablets or other forms of presentation provided in discrete units may conveniently contain an amount of one or more compounds which is effective at such dosage or as a multiple of the same, for instance, units containing 10 mg to 200 mg. The biotin-based compound and the pantothenic acid-based compound may each be administered orally in solution at a dose per day of 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 3 mM, 4 mM, 5 mM, 6 mM, 7 mM, 8 mM, 9 mM, 10 mM, or any range that includes or is between any two of the foregoing values, including fractional increments thereof.

The compounds of the disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's condition has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating an inflammatory gastrointestinal disease or disorder comprising administering to a subject having or suspected to have such a disease or disorder, a therapeutically effective amount of a composition comprising, consisting essentially of, or consisting of, a biotin-based compound and a pantothenic acid-based compound as disclosed herein or pharmaceutically acceptable salts, solvates, or prodrugs thereof. Examples of inflammatory gastrointestinal diseases and disorders, include, but are not limited to, inflammatory bowel disease, coeliac disease, ulcerative colitis, Crohn's disease, enterocolitis, gastroenteritis, enteritis, and duodenitis.

In certain embodiments, a method of treating an inflammatory gastrointestinal disease or disorder comprises administering to the subject a therapeutically effective amount of a biotin-based compound and a pantothenic acid-based compound as disclosed herein, or pharmaceutically acceptable salts, solvates, or prodrug thereof, so as to affect: (1) decreased gastrointestinal inflammation; (2) improvement in patient-reported outcomes as to the status of the patient's health condition; (3) improvement in the endoscopic appearance of mucosa; and/or (4) decreased appearance of blood in a stool.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

The compositions disclosed herein may also be combined or used in combination with other drugs and agents useful in the treatment of an inflammatory gastrointestinal disease or disorder. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more drugs or therapeutic agents selected from anti-inflammatory drugs, immunosuppressant drugs, antibiotics, tumor necrosis factor (TNF)-alpha inhibitors, biologics and pain relievers. Examples of anti-inflammatory drugs include but are not limited to, corticosteroids and aminosalicylates. Examples of corticosteroids include, but are not limited to, prednisolone, prednisone, hydrocortisone, methylprednisolone, beclometasone dipropionate, and budesonide. Examples of immunosuppressant drugs include, but are not limited to, azathioprine, mercaptopurine, cyclosporine, and methotrexate. Examples of biologics include, but are not limited to, infliximab, adalimumab, golimumab, natalizumab, vedolizumab, and ustekinumab.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating an inflammatory gastrointestinal disease or disorder in a human or animal subject in need of such treatment comprising administering to said subject an amount of a biotin-based compound and a pantothenic acid-based compound of the disclosure that is effective to reduce or prevent said disease or disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of an inflammatory gastrointestinal disease or disorder.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise a biotin-based compound and pantothenic acid-based compound, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Materials:

All chemicals and reagents used in this study were purchased from commercial vendors and were of analytical/molecular biology grade. The specific primers used for PCR amplifications were from Sigma Genosys (Woodlands, Tex.). Anti-claudin-1 (Life Technologies; catalog no. 374900) and -2 (Life Technologies; catalog no. 325600), anti-zonula occludens (ZO)-1 (Santa Cruz Biotechnology; catalog no. sc-8146), and anti-myosin light chain kinase (MLCK) (Sigma-Aldrich; catalog no. M7905) antibodies were used in these investigations.

Blood Samples:

Blood from healthy volunteers were obtained through the Institute for Clinical and Translational Science, University of California, Irvine.

Animals:

The SMVT-cKO mouse line and iSMVT-cKO mouse line were generated by using the Cre/lox technology described in Ghosal et al. (Am J Physiol Gastrointest Liver Physiol 304: G64-G71 (2013))(e.g., see FIG. 9). The animals were genotyped as described in Ghosal et al. 20-24-week-old SMVT-cKO mice were used in the experiments presented herein, and their sex- and age-matched WT-littermates as controls.

For administration of biotin and pantothenic acid, biotin (1 mM) and pantothenic acid (1 mM) were orally administered using drinking water. Doses of biotin and pantothenic acid were orally administered to mice before conception, to dams during pregnancy and lactation, and to their SMVT-cKO offspring throughout their life span as well as to WT-littermates.

Preparation of Biotin-Deficient Medium:

Serum-free AIM V medium containing gentamicin and streptomycin sulfate was purchased from Life Technologies. AIM V medium was mixed with streptavidin-agarose beads (Sigma-Aldrich, St. Louis, Mo.) at a 50:1 ratio and incubated at room temperature (RT) for 2-3 h to remove any traces of biotin, as described in Agrawal et al. (Am J Physiol Cell Physiol. 311:C386-C391 (2016)). After incubation, the medium was centrifuged at 2000 rpm for 5 min to settle the biotin-streptavidin complex. Subsequently, biotin-free medium was filtered using a vacuum-driven filtration system. This medium was considered biotin deficient, whereas the untreated medium was regarded as biotin-sufficient culture medium.

Isolation of PBMCs:

Heparinized peripheral blood collected from healthy volunteers was diluted with HBSS. Thirty milliliters of diluted blood were carefully layered over 15 mL of lymphocyte separation solution in a 50-mL conical tube. The sample was centrifuged at 2000 rpm for 20 min at RT in a swinging bucket rotor without break. The mononuclear cell layer was collected in a separate 50-mL tube and washed with 20 mL of HBSS by centrifugation at 2000 rpm for 7 min. Supernatant was discarded, and the cell pellet was resuspended in 20 mL of HBSS and washed three times by centrifugation at 1000 rpm for 5 min. The cell pellet was resuspended in the appropriate amount of PBS containing 2% FBS and used for isolation of T cells.

Isolation, Stimulation, and Culture of CD4+ T Lymphocytes:

Human CD4⁺ T cells were isolated from PBMCs using a magnetic particle-based negative-selection kit (STEMCELL Technologies, Vancouver, BC, Canada). The purity of cells obtained by this method was routinely >90%. Cells were plated in 0.5 mL of biotin-sufficient and -deficient AIMV medium in a 48-well culture plate at a density of 0.5×10⁶ cells per well and stimulated with 20 μL/mL anti-CD3/CD28 Dynabeads (Invitrogen). The culture was incubated in a humidified incubator at 37° C. supplied with 5% CO₂ for 24 and 72 h; subsequently, the supernatant was collected for cytokine analysis, and cells were harvested for further analysis.

Cytokine Analysis Using ELISA:

Levels of IFN-γ, TNF, IL-10, and IL-17 in the culture supernatant samples were evaluated using specific ELISA kits (BD Biosciences, San Diego, Calif.), as per the manufacturer's instructions. Briefly, a 96-well plate was coated with 100 μL of specific capture Ab per well and incubated overnight at 4° C. Wells were washed three times with PBS containing 0.05% Tween 20 (PBST-20) and blocked with 200 μL of 10% FBS-PBS solution per well at RT for 1 h. Wells were washed three times with PBST-20, and 100 μL of diluted sample was added per well. Serially diluted standards were also added to the appropriate wells along with the samples and incubated at RT for 2 h. Wells were washed five times with PBST-20, and 100 μL of diluted detection Ab, along with streptavidin-HRP enzyme reagent, was added per well and incubated for 1 h at RT. Wells were washed again seven times using PBST-20, with a 1-min soaking time, 100 μL of substrate solution was added to each well, and the plate was incubated at RT for 20 min in the dark. The reaction was terminated by adding 50 μL of 1 N HCl per well, and OD was measured at 450-nm wavelength using a spectrophotometer.

Estimation of Differential TFs of T Cell Subsets Using Quantitative Real-Time PCR:

Total RNA was extracted from anti-CD3/CD28-stimulated human CD4⁺ T cells cultured in biotin-sufficient and -deficient AIM V medium for 72 h using TRIzol Reagent (Invitrogen), following the manufacturer's protocol. cDNA was prepared from DNase I-treated RNA samples using an iScript kit (Bio-Rad, Hercules, Calif.). Quantitative real-time PCR was performed on a CFX96 Real-Time PCR system (Bio-Rad), as per the manufacturer's instructions, using gene-specific primers for human T-bet, GATA-3, RORγt, Foxp3, and GAPDH (as internal control) (see TABLE 1 for a list of all primers). Relative gene expression was quantified by normalizing Ct values with the corresponding GAPDH value.

Differentiation of CD4⁺ Naive and Memory T Cells:

Human CD4⁺ T cells were isolated from PBMCs using a negative-selection kit (STEMCELL Technologies). Purified CD4⁺ T cells were sorted into naive (CD4⁺CD45RA⁺CCR7⁺) and memory (CD4⁺CD45RA⁻) subsets using a BD FACSAria II cell sorter. CD4⁺ memory and naive T cells were differentiated into Th1 and Th17 subsets using CellXVivo Th1 and Th17 differentiation kits, according to the manufacturer's protocol (R&D Systems, Minneapolis, Minn.). Naive CD4⁺ T cells were differentiated into Tregs by adding 20 ng/mL each TGF-β and IL-2, along with 10 nM retinoic acid.

Naive cells were incubated in a humidified incubator at 37° C. supplied with 5% CO₂ for 6 d, whereas memory cells were cultured for 3 d. After 3 and 6 d, culture supernatant was collected for cytokine analysis, and differentiated naive cells were harvested for intracellular cytokine staining.

Intracellular Staining of IFN-γ, IL-17, and Foxp3:

Human naive and total CD4⁺ T cells were stimulated with anti-CD3/CD28 beads and cultured in biotin-sufficient and -deficient AIM V media for 3-6 d, depending on the experiments. Cells were harvested and stimulated with 1 μg/mL ionomycin and 10 ng/mL PMA (Sigma-Aldrich) for 4 h. Brefeldin A (10 μg/mL) was added to the culture at the same time. Cells were washed with PBS and surface stained with anti-human CD4 PerCP mAb (BD Biosciences) for 30 min in the dark at RT. After washing with PBS, cells were fixed and permeabilized using Cytofix/Cytoperm buffer (BD Biosciences), as per the manufacturer's instructions. Following the washing step, cells were intracellularly stained with FITC-labeled anti-human IFN-γ and Alexa Fluor 647-labeled anti-human IL-17 mAbs (BD Biosciences). F ITC-conjugated mouse IgG_{2b} and Alexa Fluor 647-conjugated IgG₁ were used as isotype controls.

For staining of Tregs, unstimulated (0 h), stimulated (72 h), and naive (6 d) cells were collected and surface stained with anti-CD4 and anti-CD25 Abs (BD Biosciences). Subsequently, cells were permeabilized using BD Foxp3 buffer, as per the manufacturer's protocol, and stained with PE-labeled Foxp3 mAbs. PE-conjugated mouse IgG1 was used as isotype control. Cells were acquired on a BD FACSCalibur flow cytometer, and data were analyzed using FlowJo software (TreeStar, Ashland, Oreg.).

Cell-Proliferation Assay Using CFSE Staining:

A total of 1×10⁶ CD4⁺ T cells per milliliter was isolated from PBMCs and stained with 2 μM CFSE dye for 10 min at RT. Cells were washed twice with PBS and stimulated with anti-CD3/CD28 Dynabeads. A total of 0.5×10⁶ cells per well was cultured in biotin-sufficient and -deficient AIM V media for 3 d at 37° C. and 5% CO₂. After 3 d, cells were collected, re-suspended in 100 μL of PBS, stained with anti-human CD4 PerCP mAb for 30 min in the dark at RT, and acquired on a BD FACSCalibur flow cytometer. Proliferation was determined by measuring CFSE dilution. Analysis was performed using FlowJo software.

For measuring cell death, CD4 Ab-stained cells were incubated with 7-aminoactinomycin D (7-AAD) for 15 min and acquired on a flow cytometer.

Induction of Dietary Biotin Deficiency in Wild-Type Mice:

Dietary biotin deficiency was induced in animals, as described in Sabui et al. (Am J Physiol Gastrointest Liver Physiol. 2016; 311:G561-G570 (2016)). Briefly, weight-matched 4-wk-old male C57BL/6J mice (The Jackson Laboratory) were divided into two groups. The first group was allowed free access to a biotin-deficient diet (containing 30% egg white), whereas the other (control) group was fed an identical amount of diet supplemented with biotin (20 mg biotin/kg food; both from Dyets). After 16 wk of pair feeding, the biotin-deficient group developed classic symptoms of biotin deficiency (which includes alopecia, dermatitis around the mouth, and decreased growth rate), whereas all mice in the control group were normal and healthy.

Histopathology Analysis in Cecum of Dietary Biotin-Deficient and Pair-Fed Wild-Type Mice:

The cecum of biotin-deficient mice and their pair-fed controls were collected immediately after euthanasia and fixed in 10% formalin overnight. Sections of the cecal wall were embedded in paraffin; H&E-stained slides were prepared by the Long Beach VAMC Histology Laboratory, as described in Ghosal et al. (Am J Physiol Gastrointest Liver Physiol. 304:G64-G71 2013)). A board-certified anatomic pathologist performed microscopic evaluation of the blinded slides.

Quantitative Real-Time PCR of Inguinal Lymph Nodes from Diet-Induced Biotin-Deficient Mice:

Inguinal lymph nodes were harvested from diet-induced biotin-deficient and pair-fed control mice. A single-cell suspension was recovered from each node, and total RNA was extracted from these cells using an RNeasy Mini Kit (QIAGEN, Santa Clarita, Calif.), following the manufacturer's protocol. cDNA was prepared from DNase I-treated RNA samples using an iScript kit (Bio-Rad). Quantitative real-time PCR analysis was performed using a CFX96 real-time PCR system (Bio-Rad), as per the manufacturer's instructions, using gene-specific primers for mouse Foxp3, RORγt, GATA-3, T-bet, and GAPDH (as internal control) (see TABLE 1 for a list of all primers). Relative gene expression was quantified by normalizing Ct values with the corresponding GAPDH value.

Estimation of Phospho-Manmmalian Target of Rapamycin and Phospho-p70S6 Kinase Expression in CD4⁺ T Cells:

Anti-CD3/CD28-stimulated CD4⁺ T cells were cultured in biotin-sufficient and -deficient media, with and without rapamycin (100 ng/mL), for 3 d. Supernatant was collected from all cells and stored for IFN-γ, IL-17, and IL-10 cytokine estimation; cells were re-suspended in 100 μL of PBS and surface stained with anti-CD4 mAb for 30 min in the dark at RT. Cells were washed and fixed with 100 μL of BD Cytofix Buffer I for 10 min at 37° C. Subsequently, cells were washed and incubated with 250 μL of ice-cold BD Phosflow Perm Buffer III for 30 min at 4° C. After washing with PBS, cells were divided into two halves. One half was stained with Alexa Fluor 647-labeled anti-human phospho-mammalian target of rapamycin (mTOR), and the other half was incubated with rabbit anti-human phospho-p70S6 kinase and then mAb for 30 min in the dark. Cells were washed with PBS and stained with PE-labeled anti-rabbit secondary Ab for 30 min in the dark at RT. Alexa Fluor 647-conjugated mouse IgG₁ was used as the isotype control. For p70S6 kinase, rabbit anti-human Ab, followed by PE-labeled secondary Ab, was used as the isotype control. After washing, cells were acquired on a flow cytometer, and data were analyzed using FlowJo software.

Estimation of Proinflammatory Cytokines and Phospho-mTOR Expression in Inguinal Lymph Nodes of Mice:

Inguinal lymph nodes were harvested from diet induced biotin deficient and pair-fed control mice. Single cell suspension was recovered from the nodes. 2.5×10⁵ cells from each sample were cultured for 12 h in biotin deficient and sufficient RPMI 1640 medium containing 1 μg/mL ionomycin and 10 ng/mL of PMA. Supernatant was collected and level of IFN-γ and IL-17 was estimated using mouse ELISA kits. Rest of the cells were surface stained with FITC labeled anti mouse CD4 mAb for 30 min in dark at RT. Cells were washed, fix and permeabilized using the same method explained above. After washing with PBS, cells were incubated with rabbit anti-mouse phospho-mTOR Ab for 30 min at RT. Following washing step, cells were stained with PE labeled anti rabbit IgG secondary Ab for 30 min at RT in dark. Rabbit anti-human Ab, followed by PE-labeled secondary Ab, was used as the isotype control for phospho-mTOR. After washing, the cells were acquired on a BD FACSCalibur flow cytometer, and data were analyzed using FlowJo software.

Intestinal Permeability Assay (FITC-Dextran Method):

Intestinal permeability was determined in vivo by measuring the appearance of FITC-dextran [molecular weight 4 kDa (FD4), Sigma-Aldrich] in the blood, as was described in Brandl et al. (Proc Natl Acad Sci USA 106:3300-3305 (2009)) and Sabui et al. (Am J Physiol Gastrointest Liver Physiol 311:G561-G570 (2016)).

Quantitative Real-Time PCR:

Total RNA was extracted from mouse tissues using Trizol reagent (Invitrogen, Carlsbad, Calif.) following manufacturer protocol and RT-PCR were performed as described in Ghosal et al. and Sabui et al. using the gene specific primers for mouse TNF-α, IFN-γ, zonula occuldens (ZO)-1, claudin-1 and -2, myosin light chain kinase (MLCK), mucin-1, mucin-2, mucin-3 and β-actin (used as an internal control for TJ protein and cytokines) and villin (used as an internal control for mucins) (see Table 1). Relative gene expression was quantified by normalizing Cₜ values with the corresponding β-actin or villin.

TABLE 1
List of primer sequences used for qPCR analysis

| Gene Name | Forward and Reverse Primer Sequences (5′-3′) |
|---|---|
| mClaudin-1 | TGTGGATGGCTGTCATTG (SEQ ID NO: 1); |
| | TGGCCAAATTCATACCTG (SEQ ID NO: 2) |
| mClaudin-2 | TTAGCCCTGACCGAGAAAGA (SEQ ID NO: 3); |
| | AAAGGACCTCTCTGGTGCTG (SEQ ID NO: 4) |
| mZO-1 | TTCAAAGTCTGCAGAGACAATAGC (SEQ ID NO: 5); |
| | TCACATTGCTTAGTCCAGTTCC (SEQ ID NO: 6) |
| mMLCK | ACATGCTACTGAGTGGCCTCTCT (SEQ ID NO: 7); |
| | GGCAGACAGGACATTGTTTAAGG (SEQ ID NO: 8) |
| mTNF-α | CATCTTCTCAAAATTCGAGTGACAA (SEQ ID NO: 9); |
| | TCGGAGTAGACAAGGTACAACCC (SEQ ID NO: 10) |
| mIFN-γ | TCAAGTGGCATAGATGTGGAAGAA (SEQ ID NO: 11); |
| | TGGCTCTGCAGGATTTTCATG (SEQ ID NO: 12) |
| β-actin | GGCTGTATTCCCCTCCATCG (SEQ ID NO: 13); |
| | CCAGTTGGTAACAATGCCATGT (SEQ ID NO: 14) |
| mMUC-1 | GGTTGCTTTGGCTATCGTCTATTT (SEQ ID NO: 15); |
| | AAAGATGTCCAGCTGCCCATA (SEQ ID NO: 16) |
| mMUC-2 | GTCCAGGGTCTGGATCACA (SEQ ID NO: 17); |
| | CAGATGGCAGTGAGCTGAGC (SEQ ID NO: 18) |
| mMUC-3 | AATGTCAGTTGCAGCGAAGT (SEQ ID NO: 19); |
| | GGAGAACACGAGGAGGATCA (SEQ ID NO: 20) |
| mVillin | CTCTCTCAACATCACCAC (SEQ ID NO: 21); |
| | TAGCCAGGACTACATAGCAG (SEQ ID NO: 22) |
| mNOS2 | CGGAGCCTTTAGACCTCAACA (SEQ ID NO: 23); |
| | CCCTCGAAGGTGAGCTGAAC (SEQ ID NO: 24) |
| mSOD-1 | GATGACTTGGGCAAAGGTGG (SEQ ID NO: 25); |
| | CTGCGCAATCCCATCACTC (SEQ ID NO: 26) |
| mFMO-2 | CAGTTTCAGACCACTGTCA (SEQ ID NO: 27); |
| | TGTATTCGCGGCTATGGA (SEQ ID NO: 28) |
| mLPO | GGGAGTGATACCTACACCA (SEQ ID NO: 29); |
| | CTAGGCTAGCATCCAGGA (SEQ ID NO: 30); |
| hT-bet | GATGTTTGTGGACGTGGTCTTG (SEQ ID NO: 31); |
| | CTTTCCACACTGCACCCACTT (SEQ ID NO: 32) |
| hGATA-3 | CTCCTCTCTGCTCTTCGCTACC (SEQ ID NO: 33); |
| | GACTCTGCAATTCTGCGAGCC (SEQ ID NO: 34) |
| hRORγt | CCCACAGAGACAGCACCGA (SEQ ID NO: 35); |
| | CCCACAGATTTTGCAAGGGA (SEQ ID NO: 36) |
| hFOXP3 | CTGACCAAGGCTTCATCTGTG (SEQ ID NO: 37); |
| | ACTCTGGGAATGTGCTGTTTC (SEQ ID NO: 38) |
| hGAPDH | CGACCACTTTGTCAAGCTCA (SEQ ID NO: 39); |
| | AGGGGAGATTCAGTGTGGTG (SEQ ID NO: 40) |
| mT-bet | TTCCCATTCCTGTCCTTCAC (SEQ ID NO: 41); |
| | CCACATCCACAAACATCCTG (SEQ ID NO: 42) |
| mGATA-3 | GGAAACTCCGTCAGGGCTA (SEQ ID NO: 43); |
| | AGAGATCCGTGCAGCAGAG (SEQ ID NO: 44) |
| mRORγt | TGAGGCCATTCAGTATGTGG (SEQ ID NO: 45); |
| | CTTCCATTGCTCCTGCTTTC (SEQ ID NO: 46) |
| mFOXP3 | CCTGCCTTGGTACATTCGTG (SEQ ID NO: 47); |
| | TGTTGTGGGTGAGTGCTTTG (SEQ ID NO: 48) |
| mGAPDH | TGTGTCCGTCGTGGATCTGA (SEQ ID NO: 49); |
| | CCTGCTTCACCACCTTCTTGAT (SEQ ID NO: 50) |

Western Blot Analysis:

For Western blot analysis, mouse tissue was homogenized in RIPA buffer (Sigma) with protease inhibitor cocktail (Roche). The soluble total protein homogenates were isolated by centrifugation at 8,000 g for 10 min, and an equal amount (45 μg) of the total proteins was loaded on a 4-12% mini gel (Invitrogen). The proteins were then transferred to a polyvinylidene difluoride membrane and probed simultaneously with mouse claudin-1 and -2, ZO-1, and MLCK antibodies (raised in mouse or rabbit) and monoclonal β-actin antibody (raised in mouse). The blots were then incubated with anti-rabbit/anti-mouse IR 800 dye and anti-mouse IR 680 dye (LI-COR) secondary antibodies (1:25,000) for 1 h at room temperature. Relative expression was quantified by comparing the fluorescence intensities in an Odyssey Infrared imaging system (LI-COR) using Odyssey application software (version 3.0) with respect to corresponding β-actin.

Estimation of Biotin Status:

Biotin status in BPS SMVT-cKO and WT-littermates was estimated as described in Bogusiewicz et al. (Am J Clin Nutr 88:1291-1296 (2008)), Lewis et al. (J Nutr 131: 2310-2315 (2001)), Ghosal et al. and Sabui et al., by measuring the total level of biotinylated proteins in the liver of these animals using western blot analysis. In these investigations, the nitrocellulose membrane was incubated first with mouse anti-β-actin antibodies. This was followed by labeling the anti-β-actin primary antibodies with anti-mouse IR 680 dye (LI-COR) and the biotinylated proteins with avidin-IR 800 dye (LI-COR). Total biotinylated proteins were analyzed using an Odyssey Infrared imaging system (LI-COR).

Histopathologic Analysis:

The cecum of SMVT-cKO mice and WT-littermate were collected immediately after euthanasia, and fixed in 10% formalin over-night and processed for staining as described in Ghosal et al. and Sabui et al.

Statistical Analysis:

BPS data presented herein are expressed as means±SE of at least three separate experiments. Significance (calculated using the Student's t-test) was set at p<0.05.

Within-group differences between biotin-sufficient and -deficient conditions were tested using a paired t test; p≤0.05 was considered significant. Each experiment was performed with at least four or five donors. Data are mean±SEM of four or five separate sets of all experiments.

Biotin Deficiency Enhances the Secretion of Proinflammatory Cytokines from CD4⁺ Cells:

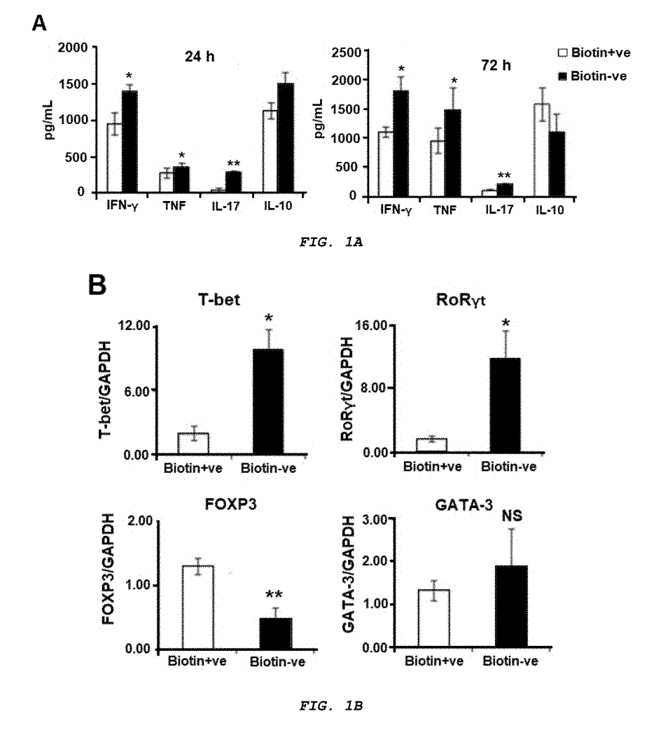

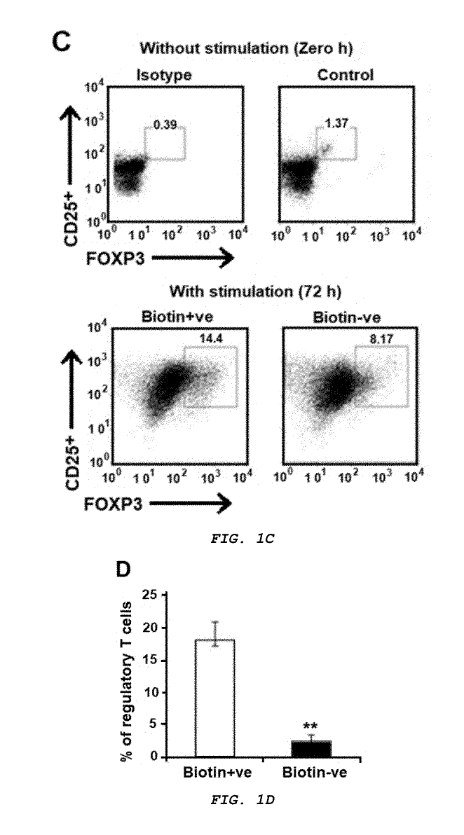

The effect of biotin deficiency on cytokine secretion by CD4⁺ T cells was determined. Anti-CD3/CD28-stimulated CD4⁺ T cells were cultured in biotin-deficient and regular media for 1 and 3 d. Supernatants were collected and assayed for IFN-γ, TNF, IL-17, and IL-10 cytokines using ELISA. As is evident from FIG. 1A, deficiency of biotin resulted in significantly increased secretion of the proinflammatory cytokines IFN-γ (p≤0.05), TNF (p≤0.05), and IL-17 (p≤0.01) from CD4⁺ T cells compared with controls at 24 h; this was increased further at 72 h (see FIG. 1A). No significant change in the level of the anti-inflammatory cytokine IL-10 was observed between the biotin-sufficient and -deficient groups (see FIG. 1A). These results suggest that biotin deficiency induces an inflammatory response in CD4⁺ T cells.

Secretion of cytokines by CD4⁺ T cells is controlled by different TFs. For example, increased Th1/IFN-γ secretion is associated with enhanced expression of T-bet, whereas RORγt expression dictates Th17/IL-17 secretion. Similarly, the TF Foxp3 is a marker of Tregs, whereas GATA-3 expression is associated with Th2 responses. Therefore, it was confirmed whether the observed increase in the induction of IFN-γ, TNF, and IL-17 under biotin deficiency also led to changes in the expression of TFs in CD4⁺ T cells. The effect of biotin deficiency on TFs was assayed on CD4⁺ T cells stimulated with anti-CD3/CD28 beads using quantitative PCR (see FIG. 1B, Table 1). The results indicate that biotin deficiency led to significantly enhanced expression of the TF T-bet (p≤0.05) compared with the biotin-sufficient condition (see FIG. 1B), whereas expression of GATA-3 was unaltered (see FIG. 1B). The level of RORγt was also increased significantly (p<0.05) in biotin-deficient CD4+ T cells compared with biotin-sufficient CD4⁺ T cells (see FIG. 1B). In contrast, the expression of Foxp3 was significantly (p<0.01) downregulated in biotin-deficient cells compared with biotin-sufficient cells (see FIG. 1B). These observations suggest that biotin deficiency leads to enhanced expression of the TFs of inflammatory Th1 and Th17 phenotypes (T-bet and RORγt), whereas the expression of Foxp3, a marker of immunological tolerance, is decreased.

Because the PCR results revealed a decrease in Foxp3 expression (see FIG. 1B), next was determined whether biotin deficiency led to a decrease in the number of Tregs. The induction of Tregs was investigated in unstimulated cells (0 h) and anti-CD3/CD28-stimulated CD4⁺ T cells (72 h), under biotin-sufficient and -deficient conditions (see FIG. 1C, 1D), using flow cytometry. The percentage of CD4⁺CD25⁺Foxp3⁺ Tregs was decreased significantly (p≤0.05) in CD4⁺ T cells cultured in biotin-deficient conditions relative to biotin-sufficient cells (see FIG. 1C). The cumulative percentage of CD4⁺, CD25⁺, and Foxp3⁺ cells was also decreased significantly (p<0.01) in biotin-deficient conditions compared with biotin-sufficient cells (see FIG. 1D). These observations suggest that biotin deficiency enhances the secretion of proinflammatory cytokines, such as IFN-γ, IL-17, and TNF, from CD4⁺ T cells and decreases the induction of Tregs.

Biotin Deficiency Enhances the Differentiation of Naive and Memory CD4+ T Cells Toward Th1 and Th17 Cells:

Next, was determined whether the observed increase in IFN-γ, IL-17, and TNF in biotin-deficient CD4⁺ T cells was a consequence of increased differentiation toward Th1 and Th17 cells. The impact of biotin deficiency was evaluated on naive and memory CD4⁺ T cell differentiation toward Th1 cells, Th17 cells, and Tregs. Naive and memory cells were cultured for 6 and 3 d, respectively, and supernatant was collected and assayed for IFN-γ, IL-10, and IL-17 cytokines (see FIG. 2). Naive cells were also stained intracellularly. Intracellular IFN-γ expression in biotin-deficient Th1-differentiated naive CD4⁺ T cells was upregulated, whereas the expression of IL-17 was not detected (see FIG. 2A). The level of IFN-γ in culture supernatants of naive and memory cells was increased significantly (p<0.05) in biotin-deficient Th1-differentiated cells, whereas IL-17 was not detected in naive cells, and no significant changes were found in memory cells (see FIG. 2A). Similarly, intracellular IL-17 expression was increased in biotin-deficient Th17-differentiated naive cells, whereas IFN-γ expression was not detected in the same conditions (see FIG. 2B). The level of IL-17 was also significantly higher (p<0.05) in the culture supernatants of memory and naive cells; however, IFN-γ was undetectable in naive cells and was comparable in memory cells (see FIG. 2B). Expression of CD4⁺, CD25⁺, and FOXP3⁺ in naive T cells cultured in biotin-deficient Treg-differentiation medium was significantly decreased (p<0.05) compared with biotin-sufficient cells (see FIG. 2C). In keeping with this, IL-10 also displayed a significant decrease (p<0.05) in naive CD4⁺ T cells in biotin deficiency, whereas IFN-γ levels were very low and comparable under both conditions (see FIG. 2C). In contrast, IL-10 levels were comparable between memory T cells cultured under biotin-deficient and -sufficient conditions, and IFN-γ exhibited a significant increase (p<0.05) in biotin-deficient conditions compared with biotin-sufficient cells (see FIG. 2C). These findings suggest that biotin deficiency enhances the differentiation of naive and memory CD4⁺ T cells toward Th1 and Th17 cells and decreases the differentiation toward anti-inflammatory Tregs.

Biotin Deficiency Decreases the Proliferation of CD4+ T Cells:

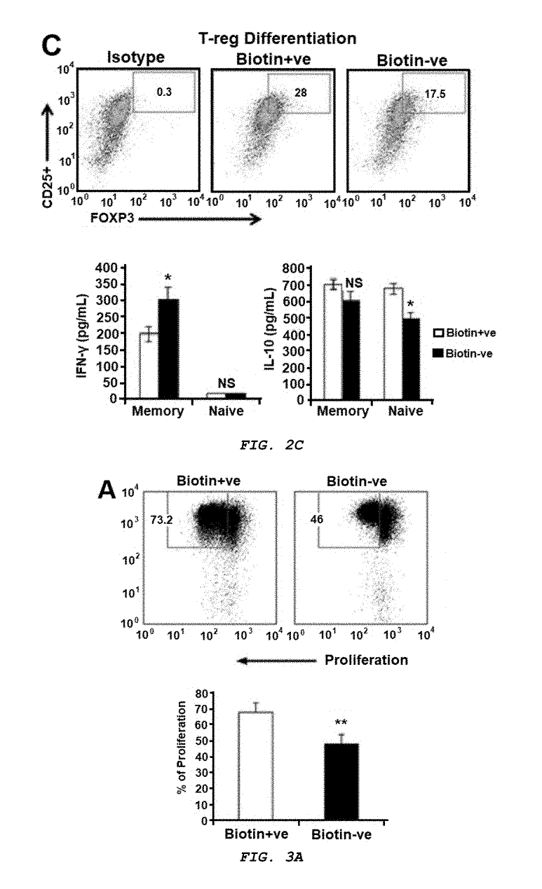

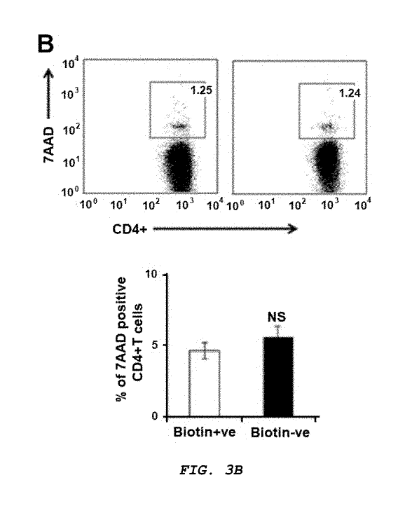

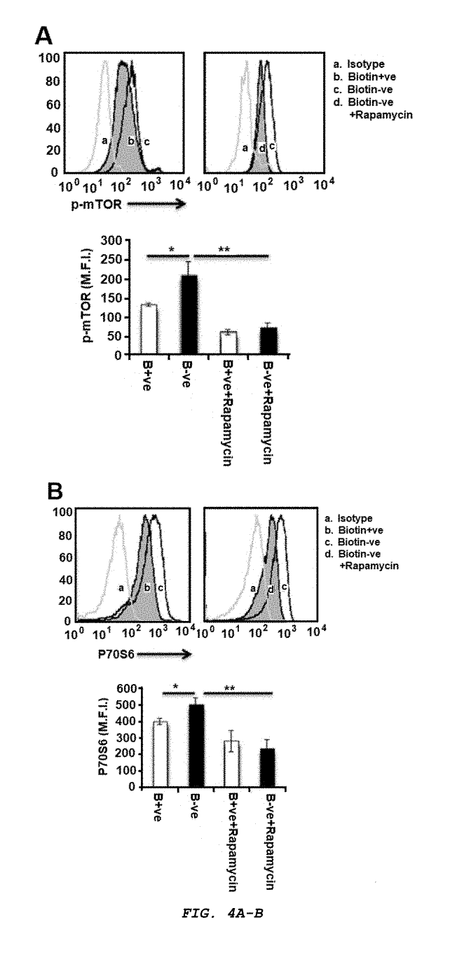

As increased differentiation of CD4+ T cells toward the Th1 or Th17 phenotype was observed, next was investigated whether biotin deficiency also enhanced the proliferation of CD4⁺ T cells to increase the inflammation. The proliferation of CD4⁺ T cells cultured under biotin-deficient and -sufficient conditions was determined by measuring CFSE dilution (see FIG. 3). The results indicate that biotin deficiency inhibited the proliferation of CD4⁺ T cells compared with biotin-sufficient cells (see FIG. 3). CD4⁺ T cells cultured under biotin-sufficient conditions underwent seven divisions (see FIG. 3). In contrast, cells cultured in biotin-deficient medium displayed initial proliferation in the first generation, but cell proliferation decreased during the subsequent divisions (see FIG. 3). The mean fluorescence intensity of each generation was also found to be lower in the biotin-deficient group compared with the biotin-sufficient group. Reduced proliferation was not a consequence of cell death under biotin deficiency, because cell viability was >90% in biotin-deficient and -sufficient conditions, as measured by 7-AAD staining (see FIG. 3B). These data indicated that biotin deficiency leads to a decrease in CD4⁺ T cell proliferation. Therefore, the increase in the production of inflammatory cytokines may be due to enhanced differentiation of CD4⁺ T cells toward the Th1 and Th17 phenotypes (see FIG. 1).

Biotin Deficiency Increases Phospho-mTOR and Phospho-p70S6 Kinase Expression in CD4+ T Cells:

Activation of T cells relies on intra- and intercellular signals from the surrounding environment, and these signals are assimilated by the evolutionarily conserved serine/threonine mTOR kinase and its downstream signaling molecule, p70S6 kinase, which regulates cell growth, proliferation, and survival. mTOR signaling has emerged as a critical inducer of inflammatory responses in CD4⁺ T cells. Because biotin deficiency enhanced the production of proinflammatory cytokines in CD4⁺ T cells, the expression of phospho-mTOR and p70S6 kinase in CD4⁺ T cells cultured in biotin-sufficient and -deficient media was evaluated (see FIG. 4). Also, the effect of the mTOR inhibitor rapamycin on the expression of these molecules in biotin-deficient cells was investigated.

The expression of phospho-mTOR was significantly upregulated in biotin-deficient CD4⁺ T cells compared with the sufficient group and was inhibited by rapamycin (see FIG. 4A). Similarly, p70S6 kinase exhibited significantly upregulated expression in biotin-deficient CD4⁺ T cells compared with the sufficient group (see FIG. 4B). Addition of rapamycin was also able to inhibit this upregulation (see FIG. 4B).

The levels of IFN-γ, IL-17, and IL-10 cytokines were also assayed in culture supernatants of biotin-deficient and -sufficient CD4+ T cells treated or not with rapamycin. Production of IFN-γ and IL-17 was significantly higher (p<0.05) in biotin-deficient CD4⁺ T cells compared with the sufficient group (see FIG. 4C). However, the levels of IFN-γ and IL-17 were significantly reduced in biotin-deficient cells treated with rapamycin compared with the biotin-deficient control group (see FIG. 4C). The level of IL-10 was comparable in biotin-deficient and biotin-sufficient cells but was reduced significantly after treatment with rapamycin (see FIG. 4C). These data indicate involvement of the mTOR-mediated signaling pathway in the increased production of proinflammatory cytokines in CD4⁺ T cells under biotin-deficient conditions.

Biotin Deficiency Enhances Inflammation in the Cecum and Alters the Expression of Th TFs and Phospho-mTOR Expression in Inguinal Lymph Nodes of Mice:

The observations were further confirmed in vivo in mice by inducing biotin deficiency through feeding of a biotin-deficient diet. As shown previously, maximum chronic active inflammation during biotin deficiency was observed in the cecum. This could be due to high infiltration of immune cells in the cecum. Therefore, the number of CD4⁺ and CD8⁺ T cells in immunostained cecum samples from were quantified from biotin-deficient mice. The number of CD4⁺ T cells was significantly increased in the cecum from biotin-deficient mice compared with the pair-fed control group (see FIG. 5A, 5B). The number of CD8⁺ T cells was also slightly increased (see FIG. 5A, 5B) in biotin-deficient mice, but this increase was less pronounced than the increase in CD4+ T cells. Neutrophil infiltration was also visible. These data suggest that CD4⁺ T cells are involved in enhancing active inflammation in vivo in biotin-deficient conditions.

Next, whether biotin deficiency induced similar changes in TFs in vivo in CD4⁺ T cells as observed in vitro in human cells was determined. Quantitative PCR of inguinal lymph nodes from biotin-deficient mice was used to determine the expression of TFs. The results indicate that the expression of TFs for Th1 and Th17 (T-bet and RORγt) was significantly (p≤0.05) upregulated (see FIG. 5C) in biotin-deficient mice. In contrast, the expression of Foxp3 and GATA-3, TFs for Tregs and Th2, was significantly (p≤0.01) decreased (see FIG. 5C) in biotin-deficient mice compared with the respective control group.

It was confirmed whether increased phospho-mTOR expression in vitro was replicated in vivo. Expression of phospho-mTOR was significantly increased in CD4⁺ T cells isolated from the inguinal lymph nodes of diet-induced biotin-deficient mice compared with pair-fed controls (see FIG. 5D). Additionally, the levels of IFN-γ and IL-17 were increased in culture supernatants of CD4⁺ T cells isolated from inguinal lymph nodes of biotin-deficient mice compared with the pair-fed control group (see FIG. 5E). These observations suggest that biotin deficiency enhances the inflammatory response via activation of the mTOR signaling pathway in CD4⁺ T cells in vitro and in vivo. Furthermore, these data indicate that the changes in the proinflammatory response of CD4⁺ T cells are not limited to the mucosa-associated intestinal immune system but are also present in the systemic distant lymph nodes.

Effect of Biotin (and Pantothenic Acid) Over-Supplementation (BPS) to SLC5A6 cKO (SMVT-cKO) Mice on Survival, Growth Rate, and General Phenotype:

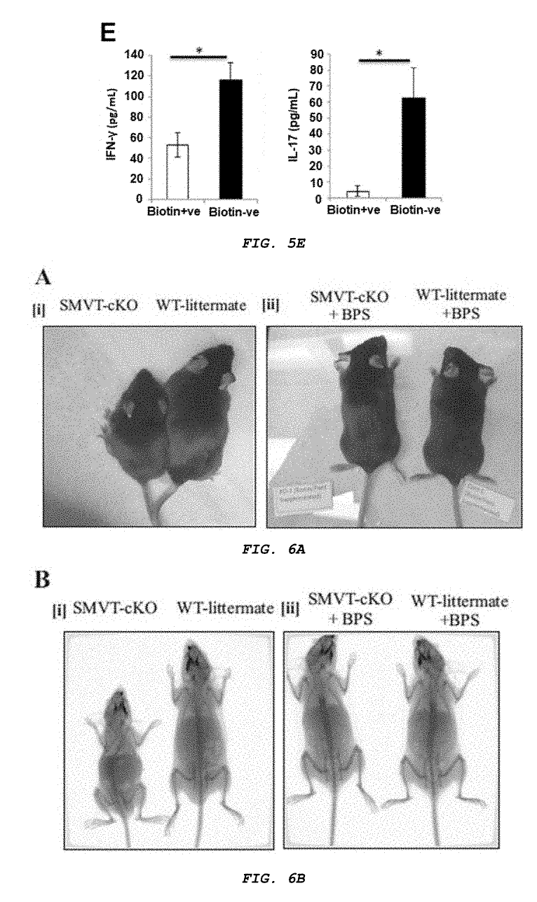

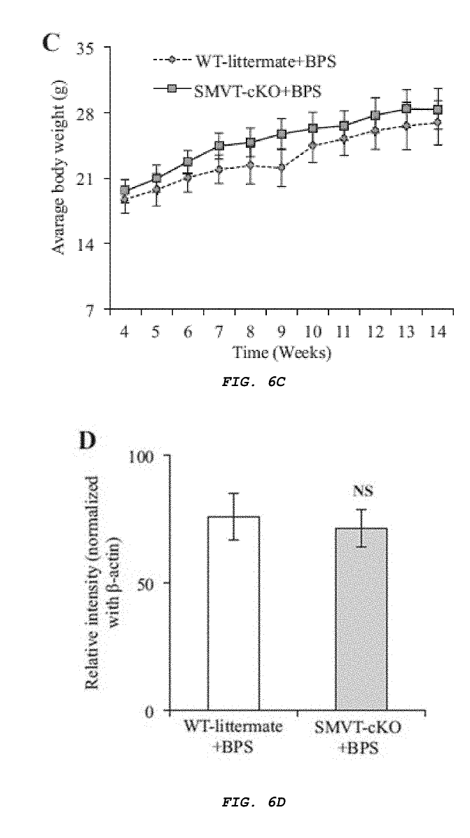

Conditional knockout of SMVT in mouse mice, SMVT-cKO, showed growth retardation (see FIG. 6A(i)), decreased bone density and length (see FIG. 6B(i)), and lethargic behavior and displayed a hunchback posture. In order to study the effects of BPS on the above abnormalities, BPS was administered to dams during pregnancy and lactation, and to their SMVT-cKO mice and sex-matched WT littermates throughout their life span. The results presented herein, indicate that administration of therapeutically effective high doses of biotin and pantothenic acid prevented early death in the SMVT-cKO animals (mice were followed for up to 6 months of age) and showed a similar phenotype to their WT-littermates. The latter includes normal growth rate, normal skeletal development, no lethargic behavior, and normal posture (see FIG. 6A-C). Also, the previously observed biotin deficiency in the SMVT-cKO animals was corrected by BPS (see FIG. 6D).

Effect of BPS to SMVT-cKO Mice on Intestinal Mucosal Morphology and Integrity:

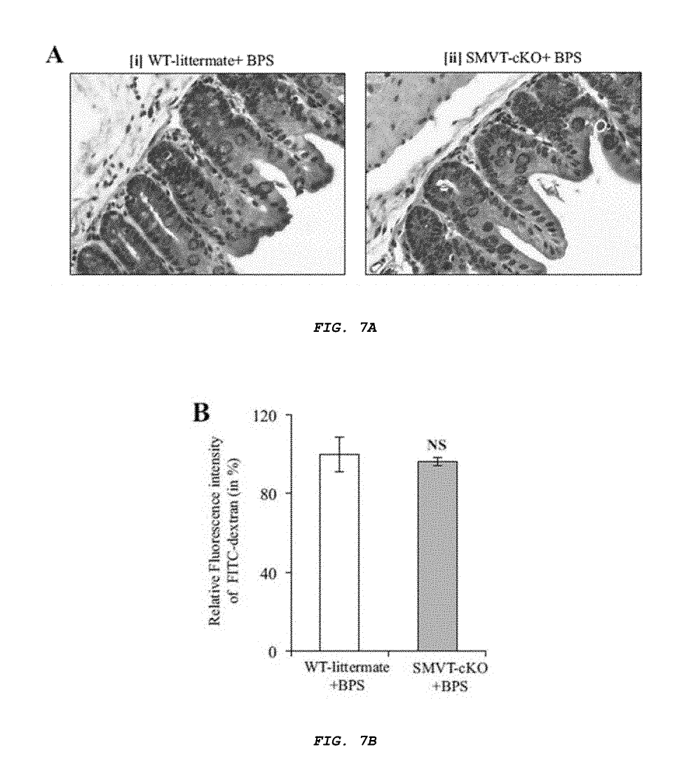

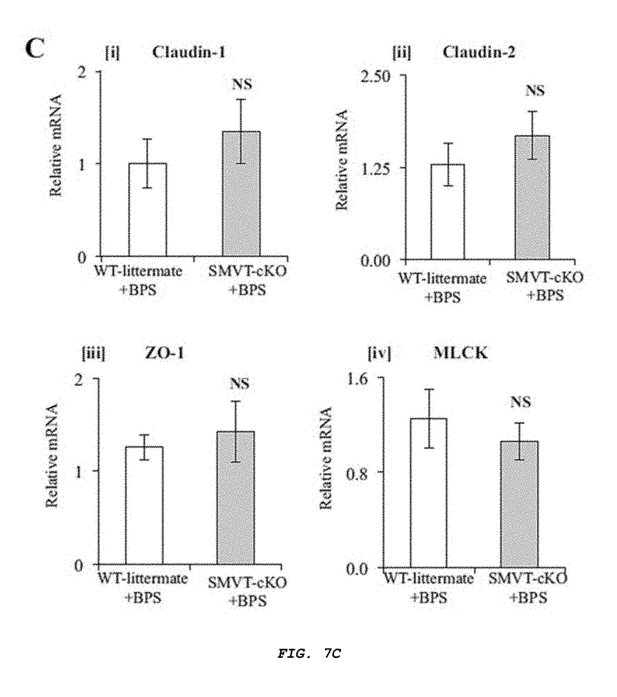

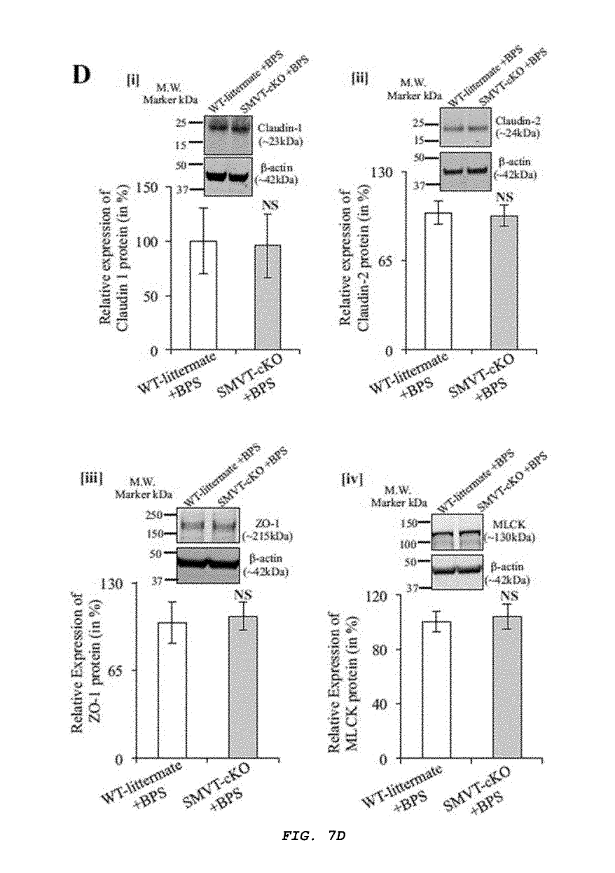

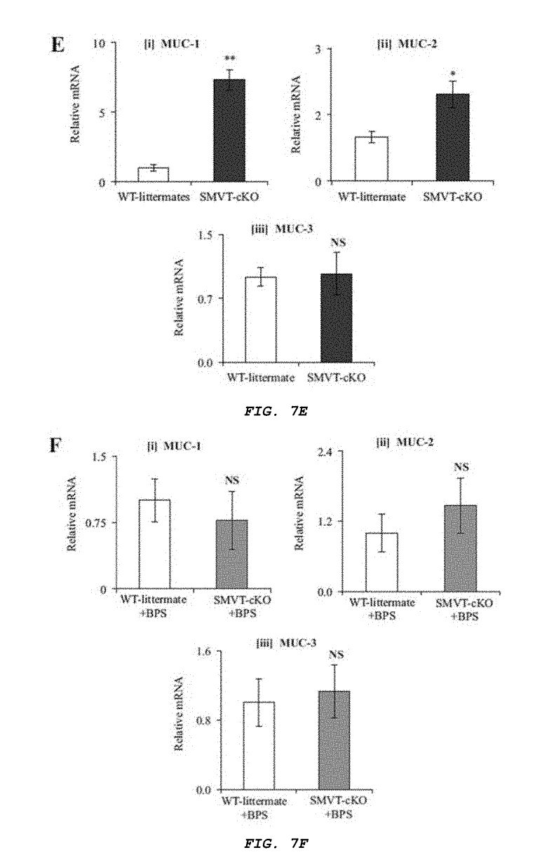

It was observed in the SMVT-cKO mice, clear histological abnormalities (neutrophil infiltration, focal cryptitis and crypt abscesses in the cecum), and an increase in gut permeability associated with marked changes in the level of expression of important TJ proteins (a significant increase in expression of the “leaky” TJ proteins claudin-1 and -2, and a decrease in the level of expression of “tight” TJ protein ZO-1, as well as a significant induction in the level of expression of TJ regulator MLCK). In this study, it was determined that high doses of biotin and pantothenic acid exerted a therapeutic benefit of preventing changes in intestinal permeability and expression of TJ proteins (and MLCK). The results presented herein demonstrated that cecal histology (e.g., see FIG. 7A) and intestinal permeability (e.g., see FIG. 7B) of SMVT-cKO mice was found to be normal and similar to those of WT-littermates. Similarly, high doses of biotin and pantothenic acid normalized the level of expression of the TJ proteins claudin-1, claudin-2 and ZO-1 as well as MLCK in the cecum (e.g., see FIGS. 7C and 7D) (and colon; data not shown) of SMVT-cKO mice similar to those seen in WT-littermates.

In other studies, the level of expression of mucin (mostly MUC-1, MUC-2, and MUC-3) was examined in SMVTcKO and whether BPS causes any observed changes. It has been postulated that aberrant expression of mucins leads to an imbalanced mucus barrier and possible inflammation that is associated with IBD and colitis. The results showed a significant increase in the level of expression of MUC-1 (P<0.01) and MUC-2 (P<0.05) (but not MUC-3) in SMVT-cKO mice (e.g., see FIG. 7E), and that this is largely normalized following administration of high doses of biotin and pantothenic acid (e.g., see FIG. 7F).

Effect of BPS to SMVT-cKO Mice on Intestinal Mucosal Inflammation:

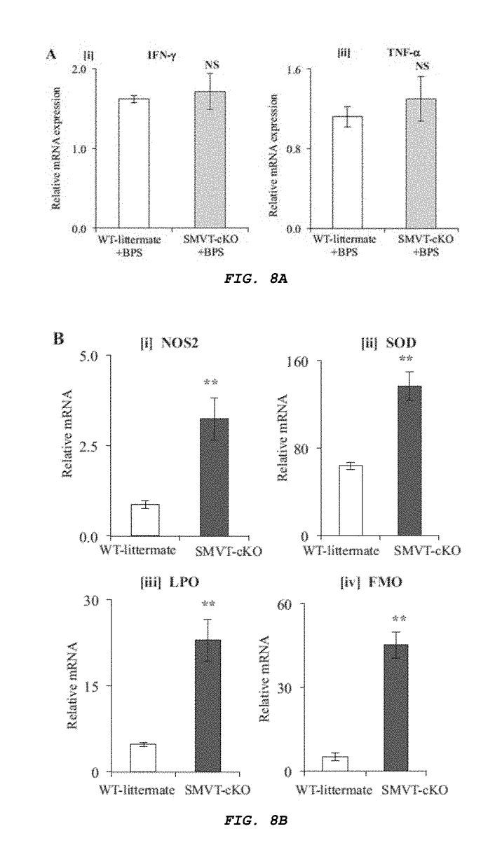

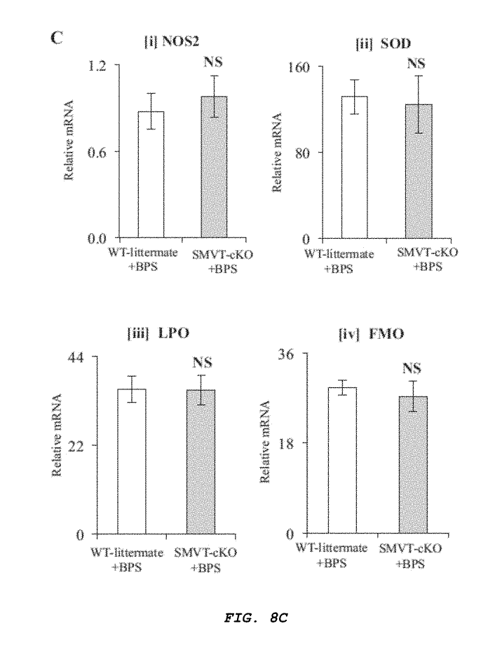

A significant induction in the level of expression of pro-inflammatory cytokines (IFN-γ and TNF-α) in the cecum of SMVT-cKO mice compared to their sex-matched WT-littermates was observed. Studies were performed to determine whether administration of high doses of biotin and pantothenic acid to SMVT-cKO mice could normalize the level of expression of IFN-γ and TNF-α in the cecal mucosa. Due to the important role that pro-inflammatory cytokines play in intestinal inflammation in IBD patients, the experiments were focused on the expression of these pro-inflammatory cytokines. The results indicate that administration of high doses of biotin and pantothenic acid normalized the level of these pro-inflammatory cytokines in the cecal mucosa of the SMVT-cKO mice to levels comparable to those in BPS-treated WT-littermates (e.g., see FIG. 8A).

Additional studies were performed to examine the level of expression of nitric oxide synthase (NOS) and reactive oxygen species (ROS) in the cecum of the SMVT-cKO mice as compared to their level in WT-littermate, and whether administration of high doses of biotin and pantothenic acid lead to changes in the levels of NOS and ROS. Active intestinal inflammation has been shown to be accompanied by an increase in the level of expression of the inducible nitric oxide synthase (NOS2) and oxidative stress-responsive genes [namely, superoxide dismutase-1 (SOD1); flavin-containing monooxygenase-2 (FMO-2); lipid peroxidation (LPO)]. The results showed a significant (P<0.01 for all) induction in the level of expression NOS2 and as well as FMO2, SOD1 and LPO in the cecum of the SMVT-cKO mice (e.g., see FIG. 8B) with complete normalization in BPS-treated SMVT-cKO mice compared to WT-littermates (e.g., see FIG. 8C).

Characterization of a Tamoxifen-Inducible SMVT Conditional (Intestine-Specific) Knockout (iSMVT-cKO) in Adult Mouse:

It was found that the isMVT-cKO mouse had significantly lower SMVT mRNA expression and SMVRT protein expression than the WT-littermates. Moreover, it was found that biotin uptake in the isMVT-cKO was significantly impaired in comparison to the WT-littermates. The iSMVT-cKO mice showed reduced body weight, biotin status and colon weight than the WT-littermates. Using hematoxylin and Eosin staining, it was found that all of the iSMVT-cKO mice developed spontaneous chronic intestinal inflammation in both the small and large intestine.

It was further found that there was a significant induction in the level of expression of the pro-inflammatory cytokines TNF-α, IL-1β, and IL-6 in the large intestinal mucosa of the iSMVT-cKO compared to Wild-type. Additionally, there was significant induction in the level of expression of the calprotectin genes was found in the large intestinal mucosa of the iSMVT-cKO mice compared to Wild-type.

By using a FITC-Dextran method, the iSMVT-cKO mice were found to have increased gut permeability. The iSMVT-cKO mice also showed changes in expression level of TJ proteins and intestinal mucin genes. All the iSMVT-cKO mice showed reduced level of expression of the Lgr5 in the intestinal tissue compared to their Wild-type littermate. Reduced expression of Lgr5 was also observed in enteroids isolated from iSMVT-cKO.

In view of the foregoing results, iSMVT-cKO mice develop biotin deficiency and a spontaneous chronic intestinal inflammation in the small and large intestine of adult mice similar to what we have seen in embryonic SMVT conditional knock out animals. The inflammation is associated with an increase in gut permeability and changes in expression of TJ proteins, mucins, calprotectin and Lgr5. Accordingly, the iSMVT-cKO mice model is an ideal model to study the relevant contribution of SMVT transporter in intestinal biotin absorption; and further supports and validates the conclusion that SMVT (biotin) plays an important role in the maintenance of intestinal health and homeostasis.

A number of embodiments have been described herein. Nevertheless, it will be understood that various modifications may be made without departing from the spirit and scope of this disclosure. Accordingly, other embodiments are within the scope of the following claims.

What is claimed is:

1. A method of treating a subject with an inflammatory gastrointestinal disease or disorder comprising administering a therapeutically effective amount of a composition consisting essentially of a biotin-based compound and a pantothenic acid-based compound.

2. The method of claim 1, wherein the biotin-based compound comprises a structure of Formula I(a): 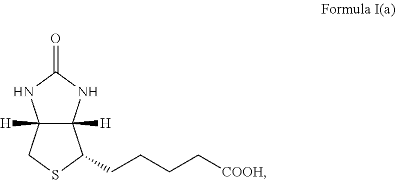 [SMILES: O=C(O)CCCC[C@@H]1SC[C@@H]2NC(=O)N[C@@H]21]
or a pharmaceutically acceptable salt, prodrug, or solvate thereof.

3. The method of claim 1, wherein the composition comprises 1 mM of the biotin-based compound.

4. The method of claim 1, wherein the pantothenic acid-based compound comprises a structure of Formula II: 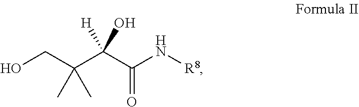
or a pharmaceutically acceptable salt, prodrug, or solvate thereof, wherein,
R⁸ is selected from the group consisting of

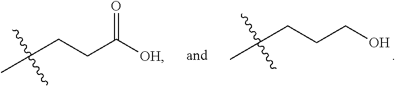 [SMILES: *CCC(=O)O ; *CCCO]

5. The method of claim 4, wherein the pantothenic acid-based compound comprises a structure of Formula II(a): 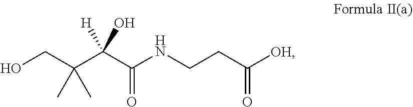
or a pharmaceutically acceptable salt, prodrug, or solvate thereof.

6. The method of claim 1, wherein the composition comprises at least 1 mM of the pantothenic acid-based compound.

7. The method of claim 1, wherein the subject is a human subject.

8. The method of claim 1, wherein the inflammatory gastrointestinal disease or disorder is selected from the group consisting of inflammatory bowel disease, coeliac disease, ulcerative colitis, Crohn's disease, enterocolitis, gastroenteritis, enteritis, and duodenitis.

9. The method of claim 8, wherein the inflammatory gastrointestinal disease or disorder is inflammatory bowel disease.

10. The method of claim 1, wherein composition consists of biotin and pantothenic acid in a pharmaceutically acceptable carrier.

11. The method of claim 1, wherein the composition is formulated for oral administration.

12. The method of claim 11, where the composition is formulated as an extended release oral formulation.

13. The method of claim 12, wherein the composition is formulated as a tablet, a capsule, a liquid filled capsule, or a gelatin-based chewable composition.

14. The method of claim 1, wherein the composition is administered with one or more drugs or therapeutic agents used to treat an inflammatory gastrointestinal disease or disorder.

15. The method of claim 14, where the one or more drugs or therapeutic agents are selected from anti-inflammatory drugs, immunosuppressant drugs, antibiotics, tumor necrosis factor (TNF)-alpha inhibitors, biologics and pain relievers.

16. The method of claim 15, wherein the anti-inflammatory drugs are selected from corticosteroids and aminosalicylates.

17. The method of claim 16, wherein the corticosteroids are selected from prednisolone, prednisone, hydrocortisone, methylprednisolone, beclometasone dipropionate, and budesonide.

18. The method of claim 17, wherein the immunosuppressant drugs are selected from azathioprine, mercaptopurine, cyclosporine, and methotrexate.

19. The method of claim 17, wherein the biologics are selected from infliximab, adalimumab, golimumab, natalizumab, vedolizumab, and ustekinumab.

20. The method of claim 1, wherein the administration of the composition provides for one of more affects selected:
(1) decreased gastrointestinal inflammation;
(2) improvement in patient-reported outcomes as to the status of the patient's health condition;
(3) improvement in the endoscopic appearance of mucosa; and/or
(4) decreased appearance of blood in a stool.


SEQUENCE LISTING (50 sequences)

SEQ ID NO: 1 - DNA, 18 bp, Artificial Sequence
  Forward Primer for mClaudin-1
tgtggatggctgtcattg

SEQ ID NO: 2 - DNA, 18 bp, Artificial Sequence
  Reverse Primer for mClaudin-1
tggccaaattcatacctg

SEQ ID NO: 3 - DNA, 20 bp, Artificial Sequence
  Forward Primer for mClaudin-2
ttagccctgaccgagaaaga

SEQ ID NO: 4 - DNA, 20 bp, Artificial Sequence
  Reverse Primer for mClaudin-2
aaaggacctctctggtgctg

SEQ ID NO: 5 - DNA, 24 bp, Artificial Sequence
  Forward Primer for mZO-1
ttcaaagtctgcagagacaatagc

SEQ ID NO: 6 - DNA, 22 bp, Artificial Sequence
  Reverse Primer for mZO-1
tcacattgcttagtccagttcc

SEQ ID NO: 7 - DNA, 23 bp, Artificial Sequence
  Forward Primer for mMLCK
acatgctactgagtggcctctct

SEQ ID NO: 8 - DNA, 23 bp, Artificial Sequence
  Reverse Primer for mMLCK
ggcagacaggacattgtttaagg

SEQ ID NO: 9 - DNA, 25 bp, Artificial Sequence
  Forward Primer for mTNF-alpha
catcttctcaaaattcgagtgacaa

SEQ ID NO: 10 - DNA, 23 bp, Artificial Sequence
  Reverse Primer for mTNF-alpha
tcggagtagacaaggtacaaccc

SEQ ID NO: 11 - DNA, 24 bp, Artificial Sequence
  Forward Primer for mIFN-gamma
tcaagtggcatagatgtggaagaa

SEQ ID NO: 12 - DNA, 21 bp, Artificial Sequence
  Reverse Primer for mIFN-gamma
tggctctgcaggattttcatg

SEQ ID NO: 13 - DNA, 20 bp, Artificial Sequence
  Forward Primer for m-beta-actin
ggctgtattcccctccatcg

SEQ ID NO: 14 - DNA, 22 bp, Artificial Sequence
  Reverse Primer for m-beta-actin
ccagttggtaacaatgccatgt

SEQ ID NO: 15 - DNA, 24 bp, Artificial Sequence
  Forward Primer for mMUC-1
ggttgctttggctatcgtctattt

SEQ ID NO: 16 - DNA, 21 bp, Artificial Sequence
  Reverse Primer for mMUC-1
aaagatgtccagctgcccata

SEQ ID NO: 17 - DNA, 19 bp, Artificial Sequence
  Forward Primer for mMUC-2
gtccagggtctggatcaca

SEQ ID NO: 18 - DNA, 20 bp, Artificial Sequence
  Reverse Primer for mMUC-2
cagatggcagtgagctgagc

SEQ ID NO: 19 - DNA, 20 bp, Artificial Sequence
  Forward Primer for mMUC-3
aatgtcagttgcagcgaagt

SEQ ID NO: 20 - DNA, 20 bp, Artificial Sequence
  Reverse Primer for mMUC-3
ggagaacacgaggaggatca

SEQ ID NO: 21 - DNA, 18 bp, Artificial Sequence
  Forward Primer for mVillin
ctctctcaacatcaccac

SEQ ID NO: 22 - DNA, 20 bp, Artificial Sequence
  Reverse Primer for mVillin
tagccaggactacatagcag

SEQ ID NO: 23 - DNA, 21 bp, Artificial Sequence
  Forward Primer for mNOS2
cggagcctttagacctcaaca

SEQ ID NO: 24 - DNA, 20 bp, Artificial Sequence
  Reverse Primer for mNOS2
ccctcgaaggtgagctgaac

SEQ ID NO: 25 - DNA, 20 bp, Artificial Sequence
  Forward Primer for mSOD-1
gatgacttgggcaaaggtgg

SEQ ID NO: 26 - DNA, 19 bp, Artificial Sequence
  Reverse Primer for mSOD-1
ctgcgcaatcccatcactc

SEQ ID NO: 27 - DNA, 19 bp, Artificial Sequence
  Forward Primer for mFMO-2
cagtttcagaccactgtca

SEQ ID NO: 28 - DNA, 18 bp, Artificial Sequence
  Reverse Primer for mFMO-2
tgtattcgcggctatgga

SEQ ID NO: 29 - DNA, 19 bp, Artificial Sequence
  Forward Primer for mLPO
gggagtgatacctacacca

SEQ ID NO: 30 - DNA, 18 bp, Artificial Sequence
  Reverse Primer for mLPO
ctaggctagcatccagga

SEQ ID NO: 31 - DNA, 22 bp, Artificial Sequence
  Forward Primer for hT-bet
gatgtttgtggacgtggtcttg

SEQ ID NO: 32 - DNA, 21 bp, Artificial Sequence
  Reverse Primer for hT-bet
ctttccacactgcacccactt

SEQ ID NO: 33 - DNA, 22 bp, Artificial Sequence
  Forward Primer for hGATA-3
ctcctctctgctcttcgctacc

SEQ ID NO: 34 - DNA, 21 bp, Artificial Sequence
  Reverse Primer for hGATA-3
gactctgcaattctgcgagcc

SEQ ID NO: 35 - DNA, 19 bp, Artificial Sequence
  Forward Primer for hROR-gamma-t
cccacagagacagcaccga

SEQ ID NO: 36 - DNA, 20 bp, Artificial Sequence
  Reverse Primer hROR-gamma-t
cccacagattttgcaaggga

SEQ ID NO: 37 - DNA, 21 bp, Artificial Sequence
  Forward Primer for hFOXP3
ctgaccaaggcttcatctgtg

SEQ ID NO: 38 - DNA, 21 bp, Artificial Sequence
  Reverse Primer for hFOXP3
actctgggaatgtgctgtttc

SEQ ID NO: 39 - DNA, 20 bp, Artificial Sequence
  Forward Primer for hGAPDH
cgaccactttgtcaagctca

SEQ ID NO: 40 - DNA, 20 bp, Artificial Sequence
  Reverse Primer for hGAPDH
aggggagattcagtgtggtg

SEQ ID NO: 41 - DNA, 20 bp, Artificial Sequence
  Forward Primer for mT-bet
ttcccattcctgtccttcac

SEQ ID NO: 42 - DNA, 20 bp, Artificial Sequence
  Reverse Primer for mT-bet
ccacatccacaaacatcctg

SEQ ID NO: 43 - DNA, 19 bp, Artificial Sequence
  Forward Primer for mGATA-3
ggaaactccgtcagggcta

SEQ ID NO: 44 - DNA, 19 bp, Artificial Sequence
  Reverse Primer for mGATA-3
agagatccgtgcagcagag

SEQ ID NO: 45 - DNA, 20 bp, Artificial Sequence
  Forward Primer for mROR-gamma-t
tgaggccattcagtatgtgg

SEQ ID NO: 46 - DNA, 20 bp, Artificial Sequence
  Reverse Primer for mROR-gamma-t
cttccattgctcctgctttc

SEQ ID NO: 47 - DNA, 20 bp, Artificial Sequence
  Forward Primer for mFOXP3
cctgccttggtacattcgtg

SEQ ID NO: 48 - DNA, 20 bp, Artificial Sequence
  Reverse Primer for mFOXP3
tgttgtgggtgagtgctttg

SEQ ID NO: 49 - DNA, 20 bp, Artificial Sequence
  Forward Primer for mGAPDH
tgtgtccgtcgtggatctga

SEQ ID NO: 50 - DNA, 22 bp, Artificial Sequence
  Reverse Primer for mGAPDH
cctgcttcaccaccttcttgat